(19) United States
(12) Patent Application Publication
(10) Pub. No.: US 2012/0101005 A1
(43) Pub. Date: Apr. 26, 2012

(54) METHODS FOR PREDICTING THE TOXICITY OF A CHEMICAL

(75) Inventors: Peter James Tatnell, Cardiff (GB); Jeffrey Kenneth Horton, Cardiff (GB)

(73) Assignee: GE HEALTHCARE UK LIMITED, LITTLE CHALFONT (GB)

(21) Appl. No.: 13/380,195

(22) PCT Filed: Jun. 23, 2010
(86) PCT No.: PCT/EP2010/003762

§ 371 Date: Dec. 22, 2011

(30) Foreign Application Priority Data
Jun. 26, 2009 (GB) 0911060.2

(51) Int. Cl. C40B 30/04; G01N 33/53; C12Q 1/02; C12Q 1/68

(52) U.S. Cl. 506/9; 435/6.12; 435/7.1; 435/29

(57) ABSTRACT

The invention relates to methods and kits for predicting the effect of a chemical on a developmental pathway. In particular, the invention relates to methods and kits for predicting the toxicity of a chemical on human developmental pathways. The methods and kits of the invention can be used for predicting changes in a cellular biomap or a developmental pathway during human foetal development.

### CROSS-REFERENCE TO RELATED APPLICATIONS

This application is a filing under 35 U.S.C. §371 and claims priority to international patent application number PCT/EP2010/003762 filed Jun. 23, 2010, published on Dec. 29, 2010 as WO 2010/149346, which claims priority to application number 0911060.2 filed in Great Britain on Jun. 26, 2009.

### FIELD OF THE INVENTION

The present invention relates to the field of cell biology, toxicology and drug screening. In particular, the invention relates to methods for predicting the toxicity of chemicals on developmental pathways.

### BACKGROUND TO THE INVENTION

The present invention describes methods designed to provide information on human cell/tissue or foetal development following chemical insult. It utilizes the in-vitro differentiation of human stem cell lines in response to known stimuli as a means to mimic in-vivo cell/tissue development. Further embodiments include i) the use of a broad range of cell/tissue markers and ii) the use of early and late cell/tissue markers. In combination these can indicate which developmental pathway is disrupted and at what stage.

During progenitor cell differentiation, cells are exposed to a chemical, drug, cosmetic or teratogen and the effect on the developmental process will be assessed by monitoring the extent of differentiation. Indeed, Suter (Current Opinion in Chemical Biology, 2006, 10, 362-366) describes the predictive value and limitations of standard (non-stem cell) in vitro tests for pharmaceutical safety evaluations. Many of the current tests have significant drawbacks because of lack of specificity of the existing methods. Indeed many existing methods are time consuming, are non-information rich, expensive and often require the use of a large numbers of laboratory animals. Thus the use of human developmental toxicity approaches yielding large amounts of data/information is an attractive alternative to traditional methods potentially reducing the number of test animals and expense without compromising the safety of consumers and patients.

Recent publications have highlighted the effects of chemicals on the developing foetus. It is estimated that ˜5% of all live births have a developmental and behavioural defect many of which are attributable to chemical insult. Furthermore the European Union has enacted (2007) a new chemical regulation called REACH (Registration, Evaluation and Authorization of Chemicals), which promises to be the most complex and comprehensive regulatory effort ever instituted. Within this law, the requirements for reproductive and developmental toxicology are very important, because they result in the highest requirement for funding and the need for experimental animals. In addition, reproductive and developmental considerations may result in the restriction of many substances that re now in widespread use. Although REACH has stringent requirements for experimental animal studies, the law discourages the use of vertebrate animals in testing, requiring laboratories to consider alternative methods. Many of the established alternative animal methods are often problematic and thus there is a great need for better and improved laboratory methods that can accurately predict the result of a chemical insult on human foetal and or reproductive development. Thus there exists a need for the introduction and validation of new cell-based methods to assess the potential toxic effect of chemicals on human development

### Stem Cells

During human development the tissues most susceptible to toxic insults include, the nervous system, liver, kidneys, lung, skin etc therefore assays using progenitor cell lines in combination with the cell/tissue specific markers will help identify not only if a particular chemical is toxic but also the cell/tissue affected. Furthermore, the approach described in this patent specification has an additional enabling feature, in that it has the capability of discriminating between early & late cell differentiation markers (for example, Nkx2.5 and αMHC are early and late cardiac markers, respectively).

Embryonic Stem (ES) cells can be differentiated, for example, into neuronal cells. Upon differentiation, expression levels of the embryonic stem cell markers and those of the differentiated cell markers can be quantified. Here embryonic stem cell differentiation is used as a means to mirror foetal/cellular development (see, for example, FIG. 1).

On chemical insult, identification of the markers in which expression levels have been disrupted facilitates the identification of which cellular developmental pathways are affected. In addition the quantification of both early and late cellular markers allows a more in-depth interrogation of the development of any particular cell type (for example, oligodendrocytes using early and late markers olig2 and MOG, respectively). Therefore quantification of all the specific cellular markers will facilitate the interrogation of the entire cellular developmental/differentiation pathways. Thus, the invention described herein can be used to construct a toxicity profile or toxicity biomap, for example for foetal development.

Stem cells are cells found in most, if not all, multi-cellular organisms. They are characterized by the ability to renew themselves through mitotic cell division and differentiating into a diverse range of specialized cell types. The two broad types of mammalian stem cells are: embryonic stem cells that are isolated from the inner cell mass of blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

Stem cells can now be grown and transformed into specialized cells with characteristics consistent with cells of various tissues such as muscles or nerves through cell culture. Adult stem cells can differentiate into developmentally unrelated cell types, such as nerve cells into blood cells. Both intrinsic and extrinsic signals regulate stem cell fate and some of these signals have been identified (Watt & Hogan, Science 2000, 287, 1427-1430).

Adult stem cells from a variety of sources, including umbilical cord blood and bone marrow, are routinely used in medical therapies. Embryonic stem cells (ES cells) are stem cells derived from the inner cell mass of an early stage embryo known as a blastocyst. Human embryos reach the blastocyst stage 4-5 days post fertilization, at which time they consist of 50-150 cells. Embryonic Stem (ES) cells are pluripotent. This means they are able to differentiate into all derivatives of the three primary germ layers: ectoderm, endoderm, and mesoderm. These include each of the more than 220 cell types in the adult body. Pluripotency distinguishes ES cells from multipotent progenitor cells found in the adult; these only form a limited number of cell types. When given no stimuli for differentiation, (i.e. when grown in vitro), ES cells maintain pluripotency through multiple cell divisions. The presence of pluripotent adult stem cells remains a subject of scientific debate; however, research has demonstrated that pluripotent stem cells can be directly generated from adult fibroblast cultures. Because of their plasticity and potentially unlimited capacity for self-renewal, ES cell therapies have been proposed for regenerative medicine and tissue replacement after injury or disease. However, to date, no approved medical treatments have been derived from embryonic stem cell research. Adult stem cells and cord blood stems cells have thus far been the only stem cells used to successfully treat any diseases.

Diseases treated by these non-embryonic stem cells include a number of blood and immune-system related genetic diseases, cancers, and disorders; juvenile diabetes; Parkinson's; blindness and spinal cord injuries. One technical problem concerning stem cell therapy is that of graft-versus-host disease associated with allogeneic stem cell transplantation. However, this problem associated with histocompatibility may be solved using autologous donor adult stem cells or via therapeutic cloning. The practical definition of a stem cell is the functional definition—a cell that has the potential to regenerate tissue over a lifetime. For example, the gold standard test for a bone marrow or hematopoietic stem cell (HSC) is the ability to transplant one cell and save an individual without HSCs. In this case, a stem cell must be able to produce new blood cells and immune cells over a long term, demonstrating potency. It should also be possible to isolate stem cells from the transplanted individual, which can themselves be transplanted into another individual without HSCs, demonstrating that the stem cell was able to self-renew. Properties of stem cells can be illustrated in vitro, using methods such as clonogenic assays, where single cells are characterized by their ability to differentiate and self-renew. Furthermore, stem cells can be isolated based on a distinctive set of cell surface markers.

### PRIOR ART

There are many published patent and patent applications relating to stem cells; where permitted, the content of those listed below are hereby also incorporated into this application by reference in their entirety.

U.S. Pat. No. 5,843,780 and U.S. Pat. No. 6,200,806 (Wisconsin Alumni Research Foundation) describes methods for isolating primate embryonic stem cells.

WO2007/120699 (Wisconsin Alumni Res Foundation) describes biomarker profiles of low molecular weight cellular metabolites (10-1500 Daltons) that are able to predict developmental toxicity and methods for screening chemical compounds including pharmaceutical agents, lead and candidate drug compounds and other chemicals using human embryonic stem cells.

Stumman et al (2009) Toxicology 257 (3) 117-126 describes embryo toxicity hazard assessment of methylmercury using embryonic stem cells to neuronal cells. This paper describes an embryonic stem cell test as a tool for developmentally toxic compounds, especially aimed to improve the in vitro prediction of methylmercury embryo toxicity. Stummann et al. describes a prediction model based on three end-points, i.e. cytotoxicity assays, RT-PCR and immunohistochemistry.

WO2007/063316 (Plasticell) discloses a method for identifying a potential modulator of a cell signalling pathway, comprising the steps of: (a) providing a cell of a first cell type, wherein the first cell type may be differentiated to a second cell type via a progenitor cell by sequentially exposing the first cell type to two or more reaction conditions; (b) adding to or replacing at least one of the two or more reaction conditions to which the progenitor cell has been exposed with exposure to one or more different reaction conditions comprising the potential modulator;—and (c) monitoring the differentiation of the first cell type to determine formation of the second cell type.

Buesen et al (2009, Toxicological Sciences, 108, (2) 389-400) describes measurement of only one biomarker (cytotoxicity) from a single sample.

WO 2004/013316 (University of Durham) discloses a method for preparing one or more compositions of clonal pluripotent stems cells derived from an individual mammalian pluripotent stem cell isolated from a cell population, comprising a heterogeneous cell population to an amount of a tag which recognises and binds individual cells that express markers of mammalian pluripotent stem cells wherein the tag comprises a retrieval means.

WO2007/002568 (Geron Corporation) describes a system for rapid determination of pharmacologic effects on target tissue types in cell populations cultured in vitro. The cells contain a promoter-reporter construct that reflects a toxicologic or metabolic change caused by the agent being screened.

U.S. Pat. No. 7,041,438 (Geron Corporation) discloses an improved system for culturing primate pluripotent stem cells in the absence of feeder cells.

US2006/0275816 (Henderson & Cheatham) describes a microarray and cell-based screening strategy that enables identification of mechanisms of pharmacology and toxicology using biomarkers.

US2004/0254736 (Michelson & Bangs) discloses methods and apparatus to identify potential toxicity in a biological system using computer modelling and biological processes.

US2002/0192671 (Castle & Elashoff) describes a method for assessing toxicity of a substance comprising: exposing a least two genes to the substance; analysing the variance of the response to the substance for each gene using contrast analysis; constructing a summary score for each gene in the set of genes; performing a logistic regression analysis upon the summary score; and using the results of the logistic regression analysis to provide a predictive model regarding the toxicity of a substance.

U.S. Pat. No. 7,354,730 (HemoGenix, Inc) discloses high-throughput assays of hematopoietic stem and progenitor cell proliferation.

U.S. Pat. No. 7,202,081 (Hoffmann La Roche) describes a method for the simultaneous determination of cell proliferation inhibition activity and cell toxicity (induction of cell death) of a substance using a proliferating mammalian cell sample as a test system.

U.S. Pat. No. 6,998,249 (Pharmacia & Upjohn) discloses methods for predicting the in vivo toxicity of a given compound. The methods involve conducting at least three distinct assays in parallel to provide information about three distinct parameters of cytotoxicity of a chemical in a given target cell, which information is useful for predicting in vivo cytotoxicity. The method does not describe techniques involving stem cells or multiplexing.

U.S. Pat. No. 6,007,993A (Insitut fur Pflanzengenetik and Kulturpflanzenforschung) describes an in vitro test procedure for the detection of chemically induced effects on embryonic development and for differentiation for the purpose of embryotoxicity/teratogenicity screening based on differentiated pluripotent embryonic stem (ES) cells from mice and rats using embryonic germ (EG) cells obtained from primoridial germ cells. The proposed test procedure is characterised in that stable transgenic ES or EG cell clones containing tissue-specific promoters and reporter genes are selected, differentiation-dependent expression of tissue-specific genes is carried out following differentiation of ES cells in the presence of embryotoxic substances acting at specific times into different germination path derivatives; this is followed by detection of chemically induced activation, repression or modulation of tissue-specific genes which regulate embryonic development.

US2008/0280300 (Plasticell) discloses a method for identifying a potential modulator of a cell signalling pathway, comprising the steps of: (a) providing a cell of a first cell type, wherein the first cell type may be differentiated to a second cell type via a progenitor cell by sequentially exposing the first cell type to two or more reaction conditions; (b) adding to or replacing at least one of the two or more reaction conditions to which the progenitor cell has been exposed with exposure to one or more different reaction conditions comprising the potential modulator;—and (c) monitoring the differentiation of the first cell type to determine formation of the second cell type.

US2008/0248503 describes methods for screening compounds to predict toxicity and residual proliferative and differentiation capacity of the lympho-hematopoietic system.

US2008/0132424 discloses a toxicity assay based on human blastocyst-derived stem cells and progenitor cells using ATP measurement for proliferation.

US2007/0248947 (Wisconsin Alumni Research Foundation) describes biomarker profiles of low molecular weight cellular metabolites and methods for screening chemical compounds including pharmaceutical agents, lead and candidate drug compounds and other chemicals using human embryonic stem cells or lineage-specific cells produced there from. The methods are for testing toxicity, particularly developmental toxicity and detecting teratogenic effects of such chemical compounds. US2007/0248947 does not describe a multiplex method or cellular biomarkers as described herein.

U.S. Pat. No. 7,541,185 (Cythera, Inc.) discloses methods for identifying one or more differentiation factors that are useful for differentiating cells in an endoderm cell population into cells which are capable of forming tissues and/or organs that are derived from the gut tube. U.S. Pat. No. 7,510,876 (Cythera, Inc) describes in vitro methods for producing human endoderm cells.

Cezar (Int. J. Pharm. Med, 2006, 20, 107-114) reviews key opportunities for stem cell technology in producing in vitro models of disease and toxic response. O'Brien & Haskins (Methods in Molecular Biology, 2007, 356, 415-425) describes multiparametric, live cell, pre-lethal cytotoxic high-content screening assays for assessing the potential of compounds for causing human toxicity. Bremer & Hartung (Current Pharmaceutical Design, 2004, 10, 2733-2747) reviews the validation of an embryonic stem cell test in comparison to in vivo results in an international blind collaborative study.

Stummann et al. (Toxicology 2007 242, 130-43) describes embryotoxicity hazard assessment of methylmercury and chromium using embryonic stem cells. This paper describes an embryonic stem cell test as a tool for developmentally toxic compounds, especially aimed to improve the in vitro prediction of methylmercury embryotoxicity. Stummann et al. describes a prediction model based on three end-points, i.e. cytotoxicity assays with mouse embryonic stem cells and 3T3 fibroblasts in addition to an embryonic stem cell cardiac differentiation assay. However, this paper does not describe the effect of toxins or teratogens on early or late differentiation biomarkers for a particular cellular developmental pathway as means to mirror foetal or cellular development.

Clarke et al. (Regen. Med. 2007, 2, 947-956) discloses the use of primary cells from various hematopoietic tissues to provide high content information. Paquette et al. (Reprod. Toxicol. 2008, 83, 104-111) describes application and use of an embryonic stem cell test as a tool for developmentally toxic compounds in the pharmaceutical industry.

Li et al. (Biol. Chem. 2008, 389,169-177) describes the effect of a toxic agent (Dioxin) inhibiting the adipogenic differentiation of an adult stem cell line. Miranda et al. (Methods Mol. Biol. 2008 447,151-156) describes the use of foetal rodent cerebral cortical-derived neural stem cells as an experimental model and determines the effect of prior ethanol exposure on the subsequent maturation of neurons.

Adler et al. (Altern Lab Anim. 2008 36, 129-40) discloses cell viability assays based on human cell types, representing different degrees of developmental maturation, namely, foreskin fibroblasts, human embryonic stem cell-derived progenitor cells, and human embryonic stem cells.

Adler et al. (Toxicology in vitro, 2008 22, 200-211) describes a developmental toxicity test method based on human embryonic stem cells and a number of marker genes.

Ahuja et al. (Toxicology 2007 231, 1-10) describes the treatment of specific cell types with chemical or physical agents; measuring their response offers a shortcut to test the toxicity in various organ systems in the adult organism.

### TECHNICAL PROBLEM

As discussed above, there is a need for new assays which can be used to predict the toxicity of chemicals on human development without revert to lengthy and costly animal tests and which more closely reflect human development rather than relying on model animal systems. In particular, there is an unmet need for assays that predict which developmental pathways and which tissues will be affected by a chemical.

### SUMMARY OF THE INVENTION

In a first aspect of the present invention, there is provided a method of predicting the toxicity of a chemical on a developmental pathway in a sample comprising the steps of:
(i) treating a control population of undifferentiated stem cells in a sample with an agent to produce a first control population of differentiated cells in a first developmental pathway;
(ii) measuring the levels of at least two biomarkers expressed in said control population of undifferentiated stems cells and/or said first control population of differentiated cells to determine control levels of expression, wherein at least one of said biomarkers is expressed at an early stage of a developmental pathway and/or differentiation and at least one of the biomarkers is expressed at a late stage of a developmental pathway and/or differentiation;
(iii) exposing a test population of undifferentiated stems cells in said sample to a chemical either before or after treating with said agent to produce a first test population of differentiated cells in a first developmental pathway;
(iv) measuring the levels of said at least two biomarkers in said test population of undifferentiated stem cells and/or said first test population of differentiated cells to determine test levels of expression;
(v) comparing said control levels of expression with said test levels of expression,
   wherein a difference in the levels of expression following exposure to said chemical is indicative of the toxicity of the chemical on said developmental pathway.

The skilled person will appreciate, for the avoidance of doubt, that the undifferentiated stem cells for use in the present invention do not include totipotent stem cells.

In a preferred aspect, step (i) of the method comprises the step of treating a population of undifferentiated stem cells with an agent to produce an n^{th} population of differentiated cells in an n^{th} developmental pathway; and repeating steps (ii) to (v) to determine a difference in the control levels of expression with the test levels of expression in said n^{th} population, wherein a difference in the levels of expression following exposure to the chemical is indicative of the toxicity of the chemical on said nth developmental pathway.

In one aspect, the first and the n^{th} developmental pathway are networked developmental pathways.

In one aspect, step (i) of the method comprises the step of treating a population of undifferentiated stem cells with an agent to produce a plurality of populations of differentiated cells in a plurality of developmental pathways; the method then comprising repeating steps (ii) to (v) to determine a difference in the control levels of expression with the test levels of expression in said plurality of populations, wherein a difference in the levels of expression following exposure to the chemical is indicative of the toxicity of the chemical on said plurality of developmental pathways.

Suitably, the plurality of developmental pathways are networked developmental pathways.

In one aspect, the stem cell is a pluripotent stem cell. The pluripotent stem cell may be an embryonic stem cell, an induced pluripotent stem cell, or a primordial germ cell.

In another aspect, the stem cell is an adult stem cell.

Preferably, the stem cell is a human stem cell.

In one aspect, at least one of the biomarkers is an embryonic stem cell biomarker.

In another aspect, at least one of the biomarkers is a primordial germ cell biomarker.

In a further aspect, at least one of the biomarkers is an adult stem cell biomarker.

Suitably the embryonic stem cell biomarker is selected from the group consisting of Nanog, SOX2, SSEA4, Oct4, TRA-1-60, TRA-1-81, Cripto, CD133, A2 B5, PAX6, Integran betal, CEA, Tnk1, ERAS and STELLAR.

Suitably the primordial germ cell biomarker is selected from the group consisting of DDX4, Fragillis, Stella and NANOS2.

In one aspect, at least one of the biomarkers is a mesodermal biomarker. Preferably, the mesodermal biomarker is selected from the group consisting of Brachyury, Tbx6, TBR2, EOMES, PHOX2A, PHOX2B, PRRX1, PRRX2, MESDC2, Mesp1, Mesp2, MIER1, MIER3 and SNAIL.

In another aspect, at least one of the biomarkers is an ectodermal biomarker. Preferably, the ectodermal biomarker is selected from the group consisting of EED, TIF1 gamma, KLH25, EDA, GJB6, ENC1, EDAR, SOSTDC1, NCAM and CD99.

In a further aspect, at least one of the biomarkers is an endodermal biomarker. Preferably, the endodermal biomarker is selected from the group consisting of Ki67, Rb, Cullin1, Cullin 2, Cullin 3, Cyclin E and Cyclin E2.

In one aspect, at least one of the biomarkers is a cardiac stem cell biomarker. Preferably, the cardiac stem cell biomarker is selected from the group consisting of hyaluronan synthase 1, OSR1 and Sca1.

In another aspect, at least one of the biomarkers is a cardiomyocyte precursor cell biomarker. Preferably, the cardiomyocyte precursor cell is selected from the group consisting of ALPK3, Periostin and Mesp 1.

In one aspect, the early biomarker is a cardiomyocyte biomarker expressed during the early stages of a developmental pathway and/or differentiation. Preferably, the early cardiomyocyte biomarker is selected from the group consisting of Nkx2.5, myocardin, GATA4, MEF2C, HAND1, IRX4, TBX5, TBX20 and Transcription Factor 25.

In another aspect, the late biomarker is a cardiomyocyte biomarker expressed during the late stages of a developmental pathway and/or differentiation. Preferably, the late cardiomyocyte biomarker is selected from the group consisting of Cardiac Traponin T antibody, Cardiac Traponin I antibody, heavy chain cardiac Myosin antibody, Myosin light chain antibody, Cardiac FABP antibody and alpha sarcomeric Actin antibody.

In a further aspect, the late biomarker is a ventricular biomarker. Preferably, the ventricular biomarker is selected from the group consisting of BMP10, HAND2 and Serum Response Factor.

In one aspect, the early biomarker is a neural stem cell biomarker expressed during the early stages of a developmental pathway and/or differentiation. Preferably, the early neural stem cell biomarker is selected from the group consisting Aggrecan ARGxxx, CD133, EMX2, Nestin and NeuroD1.

In another aspect, the late biomarker is a neural stem cell biomarker expressed during the late stages of a developmental pathway and/or differentiation. Preferably, the late neural stem cell biomarker is selected from the group consisting of BRN3A, BRN3B, Musashi 1, Msi1, NR2E1, Tailless, Nucleostemin, Oct6, Pax2, SOX2, SOX4, SOX10, SOX11, SOX22, Vimentin and CDw33.

In a further aspect, at least one of the biomarkers is a neural crest cell biomarker. Preferably, the neural crest cell biomarker is selected from the group consisting of Neurogenin 1, Neurogenin 2, Neurogenin 3 and MASH1.

In one aspect, at least one of the biomarkers is an astrocyte biomarker.

In another aspect, at least one of the biomarkers is a glial or microglial cell biomarker.

In a further aspect, at least one of the biomarkers is a Purkinja cell biomarker.

In one aspect, at least one of the biomarkers is a neuron or nerve cell biomarker. Preferably, the nerve cell biomarker is selected from the group consisting of hippochampal neuron, telencephalic neuron, dopaminergic neuron, cholinergic neuron, sensory neuron, nociceptive neuron, motor neuron, pyramidal neuron, oligodendrocytal, neuroendocrine, axons, Schwann cells, dendrites, growth cone, soma and synapse cells.

In another aspect, at least one of the biomarkers is an adipocyte biomarker. In one aspect, the levels of the two or more biomarkers are quantified by reaction with labelled antibodies and measurement of bound label. Preferably, the levels of the two or more biomarkers are quantified using quantitative immuno-cytochemistry.

In another aspect, the undifferentiated stem cells comprise different reporter genes operably linked to at least two or more biomarkers, the levels of the two or more biomarkers being quantified by measurement of the different gene products. Preferably, the reporter genes are selected from the group consisting of nitro-reductase, β-galactasidase, β-lactamase, luciferase and fluorescent protein reporter genes.

In a further aspect, the levels of the two or more biomarkers are quantified by a method selected from the group consisting of quantitative RT-PCR, quantitative immunocytochemistry, surface Plasmon resonance and microarray analysis.

In one aspect, the method additionally comprises determining cell proliferation after steps (iii).

In a preferred aspect, the method is a multiplex method.

In a second aspect of the present invention, there is provided a method of predicting changes in a cellular biomap or developmental pathway during human foetal development using the methods as hereinbefore described.

In a third aspect of the present invention, there is provided a kit for carrying out the method as hereinbefore described, the kit comprising means for quantifying at least two biomarkers and instructions for conducting the method. In a preferred aspect, the means for quantifying the biomarkers is selected from the group consisting of an antibody, an enzyme substrate and an oligonucleotide primer.

### DEFINITIONS

“Stem cells” as used herein are defined as cells that are characterized by the ability to differentiate into a diverse range of specialized cell types. The two broad types of mammalian stem cells are: embryonic stem cells that are isolated from the inner cell mass of blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

“Blastocyst” as used herein is defined as the structure formed in early embryogenesis, after the formation of the blastocoel, but before implantation. “Progenitor cells” as used herein are defined as cells having the capacity to differentiate into a specific type of cell. Most progenitors are described as unipotent or multipotent.

“Developmental pathway” as used herein is defined as a pathway for cellular differentiation (or a cellular differentiation pathway) and is the process by which a progenitor cell becomes a more dedicated cell type. Differentiation occurs numerous times during the development of a multicellular organism as the organism changes from a single zygote to a complex system of tissues and cell types. Differentiation is a common process in adults as well: adult stem cells divide and create fully-differentiated daughter cells during tissue repair and during normal cell turnover.

“Networked” in the context of “developmental pathway” as used herein is defined as a network comprising a progenitor cell that is capable of differentiating into two or more different cell types, which themselves may possess the ability to differentiate further. This process continues until a terminally differentiated cell type is generated.

“Developmental biology” as used herein is defined as the study of the process by which organisms grow and develop. Developmental biologists study the genetic control of cell growth, differentiation and morphogenesis; the process that gives rise to tissues, organs and anatomy.

“Morphogenesis” as used herein is defined as the biological process which causes an organism to develop its shape. It is one of three fundamental aspects of developmental biology along with the control of cell growth, cellular differentiation and cellular development. The morphogenesis process controls the organized spatial distribution of cells during the embryonic development and foetal development of an organism. Morphogenetic responses may be induced in organisms by hormones, by environmental chemicals ranging from substances produced by other organisms to toxic chemicals or radionuclides, pollutants, and other toxic agents, or by mechanical stresses induced by spatial patterning of the cells. Morphogenesis can take place in an embryo, a mature organism, in cell culture or inside tumor cell masses.

“Pluripotency” as used herein is defined as a stem cell that has the potential to differentiate into any of the three germ layers: endoderm (which give rise, for example to interior stomach lining, gastrointestinal tract, the lungs, liver, thymus, parthyroid and thyroid glands), mesoderm (which give rise to, for example, muscle, bone, blood, urogenital cells) or ectoderm (which give rise to, for example, epidermal tissues and the nervous system).

“Induced pluripotent stem cell” as used herein is defined as a type of pluripotent stem cell artificially derived from a non-pluripotent cell, typically an adult somatic cell, by inducing the ubiquitous expression of certain genes.

A “biomarker” as used herein is defined as a cellular molecule such as a protein, but is distinct from a low molecular weight metabolite, that is used as an indicator of a biologic state. It is a characteristic or molecule that is objectively measured and evaluated as an indicator of normal biologic processes, pathogenic processes, or pharmacologic responses to a therapeutic intervention or toxic agent. In cell biology, a biomarker is a molecule that is expressed by a particular cell type (for example, the protein Oct-4 is used as a biomarker to identify embryonic stem cells). A biomarker may be measured by a variety of techniques well known to the skilled person, such as micro-array analysis, a reporter gene assay, quantitative RT-PCR or using quantitative immunocytchemistry.

An “early” or “late” biomarker as used herein is defined as a cellular biomarker that is expressed early or during the late phases of cell culture or growth, respectively. These biomarkers may be up- or down regulated during these different phases of culture or growth.

A “multiplex assay” or “multiplexing” as used herein is defined as a type of laboratory procedure that measures multiple analytes, molecules or biomarkers from a single sample. Thus, this technique allows for the multiple interrogation of living cells allowing for the generation of high-content information. Multiplex assays are distinguished from procedures that measure single analytes or single biomarkers.

“n^{th}” as used herein denotes a positive integer from 2 to 1000 (e.g. second, third, fourth, fifth, sixth, seventh, eighth, ninth, tenth . . . one thousandth).

“Agent” as used herein is a physical stimulus (e.g. light, heat, radiation) or chemical treatment which induces differentiation of undifferentiated stem cells. The chemical treatment may comprise a mixture of chemicals, such as hormones, growth factors etc). The agent is generally different to the “chemical” which is being evaluated as a potential toxicant; where the agent and the chemical are one and the same, they are used at different concentrations in the method of the invention.

### BRIEF DESCRIPTION OF THE DRAWINGS

FIG. 1 is a schematic representation showing the differentiation of a stem cell into a network of developmental pathways (biomarkers indicated in parenthesis).

FIG. 2 is a schematic illustration of one embodiment of the method of the invention in which the effects of a chemical (20) are evaluated on the differentiation of an undifferentiated stem cell (10) to a differentiated cell (40), following treatment with a stimulus or agent (30).

### DETAILED DESCRIPTION OF THE INVENTION

### Stem Cell and Embryonic Germ Cell Markers

Stem cells are undifferentiated cells characterised by their ability to i) divide indefinably in vitro and ii) differentiate into various mature cell types. They can be categorised as pluripotent stem cells which can produce cell types of all three embryonic germ layers i.e. ecto-, meso-, and endo-dermal. These include embryonic, primordial germ cells (derived from the gonadal ridge) and reprogrammed induced pluripotent stem cells. Adult stem cells are multipotent and differentiate along a particular developmental pathway.

Emerging technologies have promised to provide the mean of identifying the markers for the various stages of stem cell differentiation. Each cell type including stem cells possesses a unique signalling network that is maintained by cell-specific transcriptional regulatory machinery which results in the expression of cell-specific genes. Therefore the 274 different cell types in the body are defined by the combinatorial expression of the ˜25,000 genes present in the human body (Ahn, S. M., et al., 2008 Proteomics 8, 4946-4957).

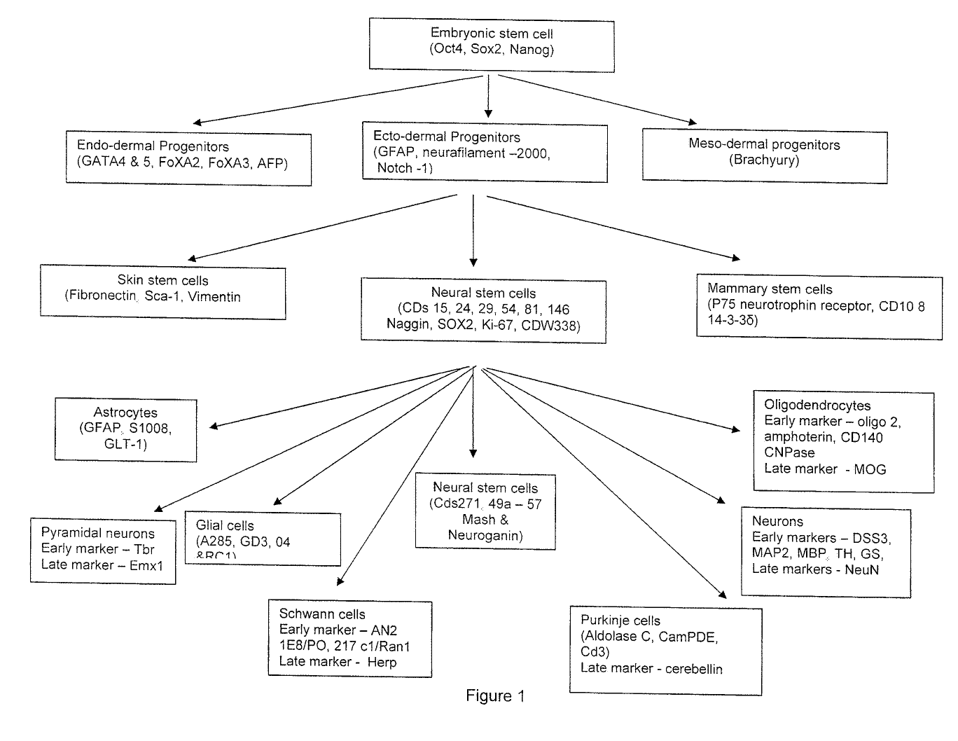

The initial stages of embryonic stem cell differentiation involves the generation of the three embryonic germ layers i.e. ecto-, meso-, and endo-dermal cells and all terminally differentiated cells are derived from these three cell types. FIG. 1 shows the differentiation of a stem cell into a network of developmental pathways. The biomarkers are indicated in parenthesis in the figure, thus Oct4 is biomarker for embryonic stem cells, while Naggin is a biomarker for neural stem cells. Early and late biomarkers are also illustrated, thus DSS3 is a biomarker which is expressed in the early stage of neuron development while NeuN is expressed at the late stages of neuron development.

In order to assess the initial extent of differentiation exhibited by a stem cell population the following germ layer markers can be used.

During later stages of differentiation more cell/tissue specific markers are used.

### Embryonic Stem Cell Markers

[1] Nanog (antibody ab21603)—Specific to early embryos and pluripotent stem cells including mouse and human embryonic stem (ES) and embryonic germ (EG) cells
[2] SOX2 (antibody ab12830)—Embryonic Stem Cell Marker
[3] SSEA4 (antibody ab16287)—Stage-specific embryonic antigen 4 is expressed early in embryonic development and in pluripotent stem cells.
[4] Oct4 (antibody ab27985)—Transcription factor expressed by undifferentiated embryonic stem cells and embryonic germ cells.
[5] TRA-1-60 (antibody ab16288)—Reacts with the antigen expressed on the surface of human tetracarcinoma and embryonic gene and stem cells.
[6] TRA-1-81 (antibody ab16289)—A marker of Human Embryonic Stem, Germ and Carcinoma Cells
[7] Cripto (antibody ab19917)—Expressed both in ES cells and during the early phases of embryo development
[8] CD133 (antibody ab19898)—A marker for stem and progenitor cells including neural and embryonic stem cells
[9] A2B5 (antibody ab53521)—A2B5 is a cell surface ganglioside epitope expressed in developing epithelial cells, oligodendrocyte progenitors and neuroendocrine cells
[10] PAX6 (antibody ab5790)—A transcription factor, important in the development of the eye, nose, central nervous system and pancreas.
[11] Integran beta1 (antibody ab5185)—Stem Cell Marker
[12] CEA Carcino embryonic antigen (antibody ab46538)—Expressed during fetal gut development
[13] Tnk1 (antibody ab70402)—A kinase Of Embryonic Stem Cells
[14] ERAS (antibody ab67696)—Expressed in embryonic stem (ES) cells and promotes their in vitro proliferation and tumorigenicity
[15] STELLAR (antibody ab78559)—A germ and embryonic stem cell enriched protein

### Primordial Germ Cell Markers

[1] DDX4 (antibody ab13840)—Primordial germ cell marker expressed in the ovary & testis.
[2] Fragillis (antibody ab15592)—Implicated in germ line cell fate
[3] Stella (antibody ab19878)—Primordial Germ Cell Marker, specifically expressed in primordial germ cells, oocytes, preimplantation embryos, and pluripotent cells.
[4] NANOS2 (antibody ab15731)—Primordial Germ Cell Marker implicated in germ cell development in both invertebrates and vertebrates.

### Mesodermal Markers

[1] Brachyury (antibody ab20680) Mesoderm Marker—The earliest indicator of mesoderm formation. Used as a marker of mesodermal differentiation.
[2] Tbx6 (antibody ab30946)—Expressed in the primitive streak and presomitic mesoderm.
[3] TBR2/Eomes (antibody ab23345)—T box brain 2 is a transcription factor that is expressed by intermediate progenitor cells during development
[4] PHOX2A and 2B (antibody ab54847 and ab12047 respectively)—Homeobox-like transcription factors involved in the development of several major neuron populations
[5] PRRX1 and 2 (antibody ab67631 and ab77655)—Members of the paired family of homeobox proteins
[6] MESDC2 (antibody ab68809)—Essential for specification of mouse embryonic polarity
[7] Mesp1 and 2 (antibody ab77013 and ab23733 respectively)—Mesoderm posterior ½ are transcription factors with a role in segmentation/patterning in the anterior pre-somitic mesoderm.
[8] MIER1 and 3 (antibody ab26254 and ab69877 respectively)—Members of mesoderm induction early response gene family.
[9] SNAIL (antibody ab17732)—Transcription factor indispensable for mesoderm formation

### Ectodermal Markers

EED (antibody ab4469)—A Polycomb-group family involved in maintaining the transcriptional repressive state of genes. Expressed during ES cell differentiation.
TIF1 gamma (antibody ab333475)—Functions in cell differentiation and development, playing a role in differentiation of hematopoietic cells.
KLH25 (antibody ab55953)—Ectodermal neural cortex protein
EDA (ab54386)—Belongs to the tumor necrosis factor family, involved in cell-cell signalling during the development of ectodermal organs.
GJB6 (antibody ab59927)—Defects cause of ectodermal dysplasias which constitute a group of developmental disorders affecting tissues of ectodermal origin.
ENC1 (antibody ab56348)—Ectoderm neural cortex protein 1
EDAR (antibody ab56803)—Ectodysplasin A receptor
SOSTDC1 (antibody ab56079)—Involved in the onset of endometrial receptivity for implantation/sensitization for the decidual cell reaction
NCAM (antibody ab6123)—Expressed on neuroectodermal derived cell lines, tissues and neoplasm such as retinoblastoma, medulloblastoma, astrocytomas and neuroblastoma
CD99 (antibody ab8855)—Expression of CD99 is a characteristic of cells from primitive peripheral neuroectodermal tumours.

### Endodermal Markers

[1] Ki67 (antibody ab833)—Ki67 antigen is the prototypic cell cycle related nuclear protein, expressed by proliferating cells in all phases of the active cell cycle
[2] Rb (antibody 2G5, ab1116)—A tumor suppressor gene, functions as a negative regulator of the cell cycle
[3] Cullin 1, 2 and 3 (antibody ab1868, ab1870 and ab1871 respectively)—mesodermal markers
[4] Cyclin E and E2 (antibody ab1108 and ab1110 respectively) Cyclin E is a regulatory subunit of Cdk2 and controls G1/S transition during the mammalian cell cycle.

### Cardiomyocyte Differentiation

Protocols to i) achieve differentiation, ii) inhibitor details and iii) cardiomyocyte markers

The differentiation of progenitor cells into cardiomyocytes is possible utilizing well characterized and published protocols such as those described by McBurney, M. W. et al., (1982), Nature 299, 165-167, Smith, S. C et al., (1987), J. Cell Physiol. 131, 74-84 and Puceat, M. 2008, Methods, 45, 168-171. Using these protocols it is possible to generate cardiomyocytes from pluri- and multi-potent progenitor cells such as embryonic carcinoma and stem cells.

### Cardiomyocyte Differentiation—Mouse P19 Derived Cardiomyocytes

P19 cells are mouse multi-potent embryonic carcinoma cells which differentiate in vitro into contracting cardiomyocytes when grown in the presence of DMSO [McBurney, M. W. (1993) Int. J. Dev. Biol. 37, 135-140]. This differentiation can be achieved by using methods described by McBurney, M. W. et al., (1982), Nature 299, 165-167 and Smith, S. C et al., (1987), J. Cell Physiol. 131, 74-84. P19 cells are commercially available from ATCC (cat. no. CRP-1825).

The “bulk culture” method described by McBurney, M. W. et al., (1982), briefly involves growing the P19 cells at 37° C. in a water-saturated atmosphere, 5.0-7.5% CO₂, in RPMI medium containing 15% (v/v) heat-inactivated foetal calf serum, 50 μg/ml streptomycin, 50 units/ml penicillin, β-mercaptoethanol (100 nM) and pyruvate (1 mM). Logarithmically growing cells are sub-cultured into differentiation medium (growth medium containing 20% heat-inactivated foetal calf serum and supplemented with 1% DMSO) at 2×104 cells per ml in a 100 mm Ultra low binding cell culture dish (Corning Cat. no. 3282). After four days the cell aggregates are transferred to a 100 mm Falcon tissue culture dish (Cat no. 353003) containing fresh differentiation medium lacking DMSO. Beating cardiomyocytes appear in the aggregates ˜6 days after exposure to the differentiation medium and DMSO.

The “hanging drop” method for differentiating P19 cells into cardiomyocytes described by Smith, S. C. et al., (1987) is similar to the bulk culture method of McBurney, M. W. et al., (1982). However, on exposure to the differentiation medium and DMSO, small volumes of cells are transferred to the roof of a 100 mm Corning Ultra low binding cell culture dish. To maintain the cells in a humidified atmosphere they are inverted over a solution of PBS. On day 4, the differentiation medium is exchanged and approximately on day 6 beating cardiomyocytes appear.

### Cardiomyocyte Differentiation—Embryonic Stem Cell Derived Cardiomyocytes

Protocols for the differentiation of the mouse embryonic stem cell lines CGR8, R1 and BS1 have been described previously Puceat, M. 2008, Methods, 45, 168-171. CGR8 and R1 are commercially available from ECACC (Cat. no. 07032901) and ATCC (Cat. no. SCRC-1011) respectively. The BS1 cell line was described and characterized by Zeineddine, D. et al., 2006, Dev. Cell 11, 535-546.

The protocol involves the generation of cellular aggregates (also known as embryoid bodies) to initiate differentiation and the use of growth factors to improve the differentiation efficiency toward a cardiac lineage. The protocol is applicable for the differentiation of both mouse and human embryonic stem cells. The major difference between propagation of mouse and human embryonic stem cells is that mouse cells can be propagated without fibroblast feeder cells in the presence of leukaemia inhibitory factor while human cells traditionally require feeder cells and FGF2 to maintain pluripotency.

Logarithmically growing mouse embryonic stem cells are exposed 24 hrs prior to commencing the differentiation process to 2.5 ng per ml recombinant human BMP2 (Invitrogen) in propagation medium. Propagation medium consists of BHK21 medium (Invitrogen), streptomycin (50 μg/ml), penicillin (50 units/ml), non essential amino acids (1 mM), sodium pyruvate (1 mM), glutamine (1 mM), mercaptoethanol (100 nM), foetal calf serum (7.5% v/v) and recombinant leukaemia inhibitory factor (1 unit per ml). On sub-culturing the cells, are dispersed (to facilitate the generation of embryoid bodies), harvested by a low speed centrifugation and re-suspended at 25,000 cells per ml in differentiation medium which consists of propagation medium lacking recombinant leukaemia inhibitory factor supplemented with 20% (v/v) foetal calf serum. All cell manipulations are performed at 37° C. and 5-7.5% CO₂.

The cells (500) are dispensed in 20 μl aliquots on to the underside of a 100 mm Corning Ultra low binding cell culture dish. PBS is dispensed into the base to prevent evaporation. Embryoid body formation is allowed to proceed for 48 hr. After which time all the embryoid bodies are gently re-suspended in 10 ml differentiation medium and incubated for a further 72 hr. On day 5, the embryoid bodies are plated onto Falcon 100 mm tissue culture dishes coated with 0.1% gelatine. Beating mouse cardiomyocytes should appear after ˜7 days.

The generation of human embryonic stem cell derived cardiomyocytes involves the following, logarithmically growing 1-6 human embryonic stem cells (Technion—Israel Institute of Technology) are cultured on E14 mouse embryonic fibroblasts using the following propagation medium—KO-DMEM (Invitrogen) supplemented with mercaptoethanol (100 nM), glutamine (1 mM), non-essential amino acids (1 mM), 15% (v/v) KOSR serum replacement (Invitrogen) and 10 ng/ml recombinant human FGF2 (invitrogen). The 1-6 human embryonic stem cell line is approved by the NIH.

To differentiate the I-6 cells into cardiomyocytes, the cells are exposed to propagation medium with a reduced concentration of KOSR serum replacement (5% v/v) and lacking FGF2 but supplemented with 10 ng/ml BMP2, and the FGF2 receptor inhibitor SU5402 (1 μM, Calbiochem Cat. no. 572630) for 48 hr.

Human embryoid bodies are generated as described above using similar protocols to those designed for differentiating mouse cells. After 1-6 cell enzymatic dissociation using collagenase CLS2 (Invitrogen) the cells are re-suspended in KO-DMEM medium supplemented with 5% KOSR serum replacement mercaptoethanol (100 nM), glutamine (1 mM) and non-essential amino acids (1 mM) and transferred to Corning Ultra low binding cell culture dish to facilitate cell aggregation. Beating human cardiomyocytes are observed after 2 weeks.

Recently a serum-free suspension culture method based upon the co-culture of human embryonic stem cells (hES2 and hES3) with mouse endoderm-like END2 cells was described by Mummery C. L. et al., 2007 Curr Protoc Stem Cell Biol Chapter 1 Unit 1F.2 and Mummery C. L. (2007) Cardiomyocyte differentiation in human ES cells. In Culture of Human Stem Cells, Chapter 4, pp 93-106 (Eds. Freshney R. I., Stacey, G. N. and Auerbach, J. M.) This protocol was further adapted by Graichen et al. 2008 Differentiation 76 357-370 for the use of END2 cell conditioned medium. Both methods involved the generation of embryoid bodies as previously described.

### Inhibitors of Cardiomyocyte Differentiation

Expression of mouse HSP25 is important for the cardiomyocyte differentiation of P19 cells. The phosphorylation of HSP25 by the p38 pathway is known to be important for certain of its functions. Inhibition of the p38 pathway by the specific inhibitor SB203580 (10 μM) has been shown to prevent mouse P19 cell differentiation into cardiomycocytes [Davidson, S. M. & Norange, M. (2000) Dev. Biol, 218, 146-160]. In this study differentiation was assessed by monitoring the presence of a single cardiac marker, cardiac-actin by immuno-histochemistry and the expression of cardiac-actin and atrial natriuretic peptide by RT-PCR.

SB 203580 [4-(4″-fluorophenyl)-2-(4″-methylsulfinylphenyl)-5-(4″-pyridyl) imidazole] is commercially available from Promega (Cat. no. V1161). It is a specific, cell-permeable inhibitor of MAP kinase homologues p38α, p38β and p38β2. SB 203580 has no significant effect on the activities of ERKs, JNKs, p38γ or p38δ.

Graichen, R. et al., (2008) Differentiation, 76, 357-370 have demonstrated that SB 203580 at 1-10 μM, actually enhances the generation of cardiomyocytes from human embryonic stem cells. However increasing the concentration to 15 μM dramatically reduces the number of cardiomyocytes and differentiation was completely blocked at 25 μM. Therefore the function of SB 203580 appears to be both species and dose dependent. These authors also demonstrated a similar concentration effect for another p38 MAP kinase inhibitor SB202190. They also reported the following inhibitors of human embryonic stem cell cardiomyocyte differentiation—SB216763 (10-25 μM, GSK-3 inhibitor), PD098059 (5-25 μM, MAPKK inhibitor), anisomycin (0.01-100 μM, JNK/SAPK and p38 activator), ATA (0.01-100 μM, JAK2/STAT5 activator), FTT 0.01-100 μM, PKC activator) and OAG (0.01-100 μM, Ca2+-dependent PKC).

Other inhibitors of cardiomyocyte differentiation include the following. The sodium/proton exchanger 1 (NHE1) inhibitor EMD87580 was demonstrated by Lei, L. et al., (2008) Shen Wu Gong Xue Bao, 24. (10), 1790-1795 to inhibit the cardiomyocyte differentiation of mouse P19 embryonic carcinoma cells during DMSO induction. Li, X. et al., (2009) Am. J. Physiol. Heart Circ. Physiol., 196, 1, 159-170 demonstrated similar results in the mouse embryonic stem cell line CGR8. The PI-3-kinase inhibitor LY294002 (50 μM) blocks the differentiation of mouse embryonic stem cells to cardiomyotes [Klinz, F., et al., (1999), Exp. Cell Res., 247, (1), 79-83].

The majority of these inhibitors are commercially available for example—SB202190 (Millipore Cat. no. 19-134), LY294002 (Promega V1201), SB216763 and anisomycin (Tocris Bioscience Cat. nos. 1616 and 1290 respectively).

The effect of inhibitors (and chemicals, drugs or cosmetics) such as SB 203580 etc., on cardiac development can be monitored during progenitor stem cell differentiation. Progenitor cells are exposed to the inhibitor and the effect on the cellular developmental process is assessed by monitoring the extent of differentiation by measuring the expression of cell/tissue specific markers associated with the differentiated cell type by techniques such as quantitative immuno-cytochemistry which are well known by the skilled person.

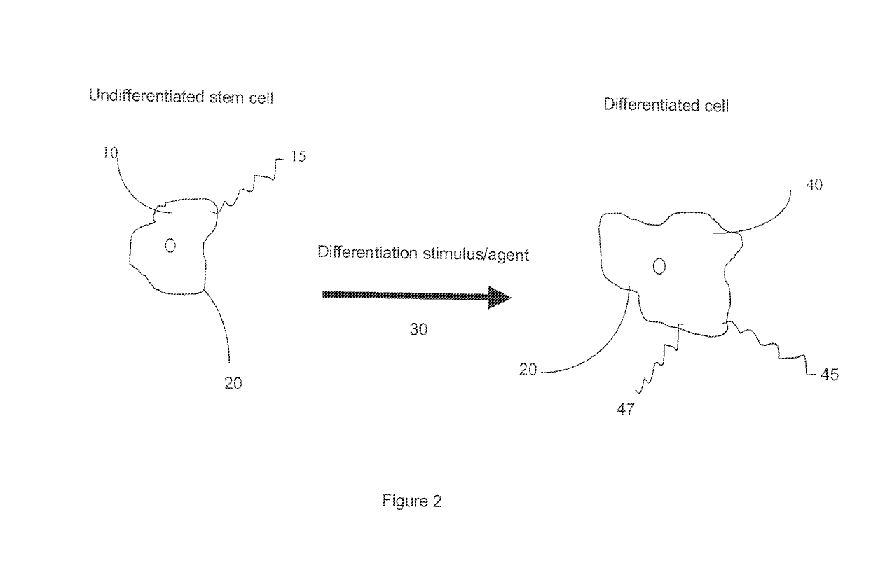

FIG. 2 illustrates one embodiment of the method of the invention in which the effects of a chemical (20) are evaluated on the differentiation of an undifferentiated stem cell (10) to a differentiated cell (40), following treatment with a stimulus or agent (30) by measuring the levels of at least two biomarkers (15, 45 and 47). The stem cell (10) may be exposed to the chemical (20) before or after exposure to a stimulus or agent (30) which induces differentiation. Thus, for example, in the context of cardiomyocyte development, the potential effects of a drug or chemical (20) on the differentiation of a stem cell (10) into a cardiomyocyte (40) can be evaluated by measuring the levels of at least two biomarkers present in the stem cell (15) or cardiomyocyte (45, 47).

Markers indicative of cardiac development include the following, (the source of antibodies commercially available from Abcam Inc. are also described):

### Cardiac Stem Cell Markers

[1] Hyaluronan synthase 1 (antibody ab75329)—Implicated in heart development.
[2] OSR1 (antibody ab76689)—A transcription factor that is expressed in the mesendoderm and subsequently in the endoderm and intermediate mesoderm.
[3] Sca1 (antibody D7, ab25031)—It is expressed on multipotent hematopoietic stem cells.

### Markers of Cardiomyocyte Precursor Cells

[1] ALPK3 (antibody ab57526)—Plays a role in cardiomyocyte differentiation.
[2] Periostin (antibody ab14041)—Expressed in the embryonic and fetal heart, localized to the endocardial cushions that ultimately divide the primitive heart tube into a four-chambers.
[3] Mesp1 (antibody ab77013)—Mesp1 is expressed in many of the precursors of the cardiovascular system and is known to play a role in the process of cardiac morphogenesis

### Cardiomyocyes—Early Markers

[1] Nkx2.5 antibody (Abacm—ab35842)—Expressed in both the myocardium and the endocardium.
[2] Myocardin antibody (Abcam Inc.—ab22621)—Myocardin regulates the expression of a set of cardiac and smooth muscle-specific genes. It plays a crucial role in cardiogenesis and differentiation of the smooth muscle cell lineage.
[3] GATA4 antibody (Abcam Inc.—ab61170)—A transcription factor involved in cardiac development, lays a role in regulating basal, agonist or stress induced gene expression in cardiac and smooth muscle cell types.
[4] MEF2C (antibody ab43796)—A transcription activator controlling cardiac morphogenesis and myogenesis, and is also involved in vascular development. It may also be involved in neurogenesis and in the development of cortical architecture
[5] HAND1 (antibody ab52767)—A transcription regulator, plays an essential role in early cardiac morphogenesis. In the adult, it is required for expression of cardiac-specific genes.
[6] IRX4 (antibody ab56032)—Expressed in the heart during development.
[7] TBX5 (antibody (ab18531)—TBX5 may play a role in heart development.
[8] Tbx20 (antibody ab42468)—Expressed in the developing heart.
[9] Transcription factor 25 (antibody ab67762)—Acts as a transcriptional repressor of SRF in vitro and hence may play a role in heart development.

### Cardiomyocyes—Late Markers

[1] Cardiac Troponin T antibody (Abcam Inc.—1C11, ab8295)—Expressed only in myocardium, cardiac Troponin T is the tropomyosin binding subunit of the troponin complex.
[2] Cardiac Troponin I antibody (Abcam Inc.—28419C7, ab19615)—Troponin I is part of a heteromeric complex playing an important role in the regulation of skeletal and cardiac muscle contraction.
[3] Heavy chain cardiac Myosin antibody (Abcam Inc.—3-48, ab15)—Cardiac MHC exists as two isoforms, alpha-cardiac and beta-cardiac MHC. Both are expressed in the human heart, beta-cardiac MHC being the predominant form.
[4] Myosin light chain antibodies (& 1LC-14, ab50080)—Myosin consists of two heavy chains and four light chains. Ventricle myosin light chain I (Abcam Inc.—MLM527, ab680) and Cardiac Myosin light chain 11 LC-14, ab50080
[5] Myosin Light Chain 2 antibody (Abcam Inc.—ab48003)—Myosin Light Chain 2 is associated with cardiac myosin beta heavy chain.
[6] Cardiac FABP antibody (Abcam Inc.—67D3, ab16916)—Expressed in cardiac muscle tissue and in significantly lower concentration in skeletal muscles.
[7] alpha sarcomeric Actin antibody (5C5, ab7799)—Alpha actin is one of the isoforms of Actin. In vertebrates there are three groups of actin isoforms: alpha, beta and gamma. The alpha actins are found in muscle tissues and are a major constituent of the contractile apparatus. This antibody reacts with a-cardiac muscle actin.

### Ventricular Markers

[1] BMP10 (antibody ab34962)—An essential component in modulating cardiomyocyte proliferation and maturation during cardiac ventricular development.
[2] HAND2 (antibody ab56590)—Expressed in the developing ventricular chambers and play an essential role in cardiac morphogenesis.

### Sarcomere Markers

[1] Sarcomeric Alpha Actinin antibody (Abcam Inc.—EA-53, ab9465)—ACTN2 encodes a muscle-specific, alpha actinin isoform that is expressed in both skeletal and cardiac muscles. Located at Z lines and dots in stress fibers of myotubes in cardiac and skeletal muscle.
[2] Serum Response Factor (antibody ab36747)—A cardiac enriched transcription factor that is required for the appearance of beating sarcomeres in the heart.

### Other Cardiac Markers

[1] HEY2 (antibody ab70133)—Transcription factor, an important determinant of mammalian heart development.
[2] KLF13 (antibody ab15701)—Transcription factor, required for heart development.
[3] MEF2 family of transcription factors MEF2A (antibody ab55547)—Has key roles in cardiac and skeletal muscle development
MEF2B (antibody ab55565)—Regulate expressions of many muscle related genes during development. Involved in differentiation of certain neurogenic cells.
MEF2D (antibody ab43797)—Present in undifferentiated myoblast suggesting that it may play a role in the very early stages of myogenic development. Involved in the differentiation of myogenic and also some neurogenic cells.
[4] Phospholamban antibody (Abcam Inc.—2D12, ab2865)—Regulates the calcium pump of cardiac sarcoplasmic reticulum (SR).

Methods described above and variations thereof are routinely used to differentiate progenitor cells such as embryonic stem and carcinoma cells into cardiomyocytes. This differentiation process can be inhibited by chemicals such as SB 293580. A combination of the various differentiation methods, characterised inhibitors and the antibodies would form the basis of a multiplex quantitative immuno-cytochemical method for assessing the effect on mammalian cell/tissue development after chemical insult.

### Neural Differentiation

Protocols to i) achieve differentiation, ii) inhibitor details and iii) neural markers

The differentiation of progenitor cells into cells of the neural lineage is possible utilizing well characterized and published protocols such as those described below. Using these protocols it is possible to generate neuronal cells from pluri- and multi-potent progenitor cells such as embryonic carcinoma and stem cells.

Embryonic stem (ES) and carcinoma (EC) cells satisfy many of the criteria for studying neuronal differentiation in that they both divide rapidly and are able to differentiate into a variety of cell types including neurons. ES cells are pluripotent however, they require demanding culturing conditions often requiring feeder cells or expensive growth factors. EC cells however, are easier to culture, do not require feeder cells and can be maintained in relatively simple media. The disadvantages of EC cells are that they are tumour cells, genetically abnormal and exhibit a limited capacity for differentiation. However, they do represent a simple and robust system for differentiation studies.

The EC cell line NTERA2 has been used for studying neuronal differentiation and exposure to retinoic acid reliably generates neurons similar to those produced by primary neurons in culture. Retinoic acid exposure results in the loss of stem cell markers such as Oct4, SSE3, TRA-1-60 and TRA-1-81 and the up regulation of neural markers such as NeuroD1, β-III tubulin and neurofilaments. This differentiation process is very predictable and consistent. Exposure to retinoic acid and the re-seeding of cells in the presence of mitotic inhibitors facilitates the generation of an essentially pure population of neurons (Leypoldt F., et al., 2001, Neurochem. 76, 806-814). These are essentially functionally mature expressing synapses's and neurotransmitter phenotypes such as cholinergic, GABAergic and sertonergic receptors (Hartley et al., 1999, J. Comp. Neurol., 407, 1-10).

### The Neural Differentiation of Progenitor Cells

The neural differentiation of NTERA2 cells as performed by Leypoldt F., et al., 2001, (Neurochem. 76, 806-814) briefly, involved the following. Cells were routinely maintained in OptiMEM (Invitrogen) medium supplemented with 5% foetal calf serum at 37° C. in 5% CO₂. Cell aggregation (1×10⁶ cells/ml) was performed in bacteriological plates in high glucose DMEM (Invitrogen) with 10% foetal calf serum. After overnight the medium was supplemented with 1 μM trans-retinoic acid. The medium and culture dishes were replaced every 3 days. The presence of retinoic acid was maintained for 21 days after which the cell aggregates were transferred to poly-D-lysine and laminin (both at 10 μg/ml) cell culture treated dishes in aggregation medium [supplemented with the mitotic inhibitors cytosine-D-arabinofuranosid (10 μg/ml) and uridine (1 μg/ml)]. These conditions were continued for ˜7 days, the medium being changed every 2-3 days. This prolonged protocol (lasts ˜4 weeks) facilitates the efficient differentiation of a high percentage of the NTERA2 cells into neurons.

Neuronal cells expressing functional synapses have also been generated from NTERA2 cells. Hartley et al., 1999, (J. Comp. Neurol., 407, 1-10) co-cultured NTERA2 cells with primary astrocytes and the resultant neurons generate glutamatergic and GABAergic synapses, (in addition their in vitro viability was extended to >1 year). Primary astrocytes were isolated from the cerebral hemispheres of 18-21 day rat embryos according to the method of Cadelli, D. S. and Schwab, M. E., 1991, (Ann. N.Y. Acad., Sci., 633, 234-240). NTERA2 neurons (1×10⁵) were seeded on poly-D-lysine and Matrigel™ (BDbiosciences) coated cover-slips and co-cultured with (but not in contact with) confluent astrocytes in a 35 mm cell culture-treated dish. The co-culture was maintained in DMEM with high glucose supplemented 1:1 with astrocytes-conditioned medium. After 6 weeks electron micrographs indicate the presence of synapses and synapsin I expression was demonstrated by immunohistochemistry. Gluatamatergic, GABAergic and NMDA transmission was confirmed by electrophysiology using the selective antagonists CNQX (10 μM), bicuculline (20 μM) and APV (100 μM) respectively.

Undifferentiated human ES cells (like NTERA2 cells) express the markers Oct4, SSE3, TRA-1-60 and TRA-1-81 all of which are down-regulated on differentiation. On neuronal differentiation with retinoic acid, dimethyl sulfoxide (DMSO) or hexamethylenebisacetamide, the derivative ES cells exhibit the down-regulation of ES markers and an increased expression of the neural ganglioside glycolipids GD2, GD3 and A2B5 (Draper J., et al., 2002, J. Anat., 200, 249-258). These cell surface markers are often used to isolate prospective neural precursors from early differentiating cells. Other useful markers for the isolation of cells from the neural lineage include N-CAM, neuroD1, NSE, nestin β-tubulin and musashi-1. Oligodendrocytes can be identified using the markers Olig-1, -2-3 and -4 (Jackson J. P., et al., 2007, Techniques for neural differentiation of human EC and ES cells. In, Culture of human stem cells eds. Freshney R. I., Stacey, G. D. & Auerbach J. M. J.Wiley & Sons, Inc.).

Methods to promote the neural differentiation of progenitor cells include cell aggregation. This method is routinely used for inducing the differentiation of ES and EC cells (including NTERA2, P19 and PC12). Cell aggregation increases cell contact, promoting intercellular signalling which is an important aspect of in-vivo development and in-vitro differentiation. Early neural differentiation techniques utilised retinoic acid in serum-containing medium but these are now replaced by better defined serum-free methods.

### Neural Differentiation—Serum-Free Defined Medium

The generation of neurospheres from EC and human ES cells by the cell aggregation technique using serum-free defined medium has been shown to produce radial glial cells from NTERA2 cells (Marchal-Vitorion S., et al., 2003, Cell Neurosci., 24, 198-213). This is a multistage process and the neurospheres can be maintained indefinitely if the medium is supplemented with FGF-2. Using human ES cells, on FGF-2 withdrawal the neurospheres differentiate into astrocytes, neurons and oligodendrocytes (Zhang, S. C. et al., 2001, Nat. Biotechnol. 19, 1129-1130).

To generate neurospheres Marchal-Vitorion S., et al., 2003, Cell Neurosci., 24, 198-213 used NTERA2 cells grown in 25 cm² flasks (1×10⁵ cells/ml) containing a serum free-defined medium DMEM/F12, (Invitrogen) supplemented with N2 (Life Technologies), glutamine (2 mM), glucose (0.6% w/v), insulin (20 μg/ml), heparin (2 μg/ml), EGF (20 ng/ml) and FGF (10 ng/ml). To induce neural differentiation, the resultant cell aggregates or neurospheres were dissociated and seeded at 5×10⁵ cells/cm² on poly-D-lysine-coated dishes and cultured for an additional 10 days in the serum-defined medium minus the growth factors. These NTERA2 neurospheres were subsequently shown to generate a large percentage of immature neurons (˜50%) together with cells of the oligodendrocyte lineage.

Using the human ES cell lines H1 and H9 grown on an inactivated mouse embryonic fibroblast feeder layer Zhang, S. C. et al., 2001, (Nat. Biotechnol. 19, 1129-1130) generated neural precursor cells that exhibited neural-like structures in the absence of serum but in the presence of FGF-2. On removal of the FGF-2 they differentiated into neurons, astrocytes and oligodentrocytes. Routine maintenance of the ES/fibroblast co-culture was performed in ES cell medium consisting of DMEM/F12 medium supplemented with 20% v/v serum replacement medium (Invitrogen), β-mercaptoethanol (0.1 mM), heparin (2 μg/ml) and FGF-2 (4 ng/ml)]. To induce differentiation the ES cell colonies were removed using Dispase™ (0.1 mg/ml Invitrogen) and resuspended in the defined ES cell medium minus FGF-2. The cells were cultured as floating embryoid bodies in a 25 cm² cell culture flask with a daily medium change. After 4 days, the embryoid bodies were resuspended in DMEM/F12 supplemented with insulin 25 (μg/ml), transferrin (100 μg/ml), progesterone (20 nM), putrescine (60 uM), sodium selenite (30 nM), heparin (2 μg/ml) and FGF-2 (20 ng/ml). The differentiating embryoid bodies were cultured for ˜10 days and then transferred to a new flask coated with poly-(2-hydroxyethyl-methacrylate) to prohibit attachment.

To generate oligodentrocytes the ES-cell derived neural precursor cells were culture in the absence of FGF-2 in DMEM supplemented with N1 (Invitrogen) and PDGF-1 (2 ng/ml). After ˜2 weeks olig4-positive cells were observed possessing typical oilgodentrocytic morphology.

Neuronal differentiation was performed by culturing the cells on ornithine/laminin in a medium consisting of DMEM/F12, N2 (Invitrogen), cAMP (100 ng/ml) and BDNF (10 ng/ml) in the presence of FGF-2 (20 ng/ml). After ˜10 days, fibre processes were observed emanating from the adherent spheres. The majority of the cell expressed the neuronal markers MAP2ab and β-tubulin.

### Neural Differentiation—Co-Culture

Additional efforts to increase the efficiency of ES and EC neural differentiation has utilised the co-culture with other cell lines e.g. the PA6 stromal cells (Scwartz et al., 2005, Stem Cell Dev., 14, 517-534). The authors co-cultured NTERA2 cells (at 2000 cells/ml) on a confluent monolayer of PA6 cells. The PA6 cells were inactivated with 10 μg/ml mitomycin-C. The co-culture was maintained in a differentiation medium similar to that described earlier (Leypoldt F., et al., 2001, Neurochem. 76, 806-814). After 22 days tyrosine hydrolase was detected in 86% of the NTERA2 neurons indicating the presence of a mature dopaminergic phenotype. The use of PA6 stromal cell conditioned media was shown to be less effective at generating the dopaminergic phenotype as the factor(s) that facilitates the differentiating process are still not fully characterised.

### Neural Differentiation—Monolayers

Other neural differentiation methods have been based on the differentiation of human ES cells in monolayers (Gerrard, L., et al., 2005, Stem Cell, 23, 1234-1241). These studies have shown that the addition of the BMP inhibitor noggin to ES cell cultures results in the generation of neural progenitor cells. The method utilised growing ES cells on Matrigel™ (BD Biosciences) in mouse embryonic fibroblast conditioned medium supplemented with FGF-2 (20 ng/ml). For neural differentiation, confluent ES cells were cultured in N2B27 neural differentiation medium (Invitrogen) supplemented with 100 ng/ml noggin. At passage 3 the cells were disassociated into single cells and cultured in N2B27 supplemented with FGF-2. This protocol resulted in the generation of neural progenitors with ˜97% of the cells expressing the neural marker musashi. Tyrosine hydrolase expressing neurons and neural progenitor cells were generated by seeding the N2B27-treated cells onto poly-L-lysine/laminin-coated dishes and cultured for 2 weeks in N2B27 medium supplemented with sonic hedgehog (300 ng/ml), FGF-8 (100 ng/ml) and ascorbic acid (160 μM). After 2 weeks sonic hedgehog was withdrawn and replaced by BDNF (20 ng/ml), GDNF (20 ng/ml), ascorbic acid (160 μM) and laminin 500 ng/ml).

A detailed description of several of the many neural differentiation methods described in this document is presented in Jackson J. P., et al., 2007, Techniques for neural differentiation of human EC and ES cells. In, Culture of human stem cells eds., Freshney R. I., Stacey, G. D. & Auerbach J. M. J.Wiley & Sons, Inc.). Example protocols described include the induction of human EC cell neural differentiation by retinoic acid, Human ES cell neural differentiation in Embryoid bodies, and the derivation and differentiation of neurospheres from human Es cells.

### Example 1

### Induction of Human EC Cell Neural Differentiation by Retinoic Acid

NTERA2 cells are maintained in growth medium (DMEM, 4.5 g/l glucose and 10% v/v foetal calf serum) at 37° C. in 10% CO₂ in air. NTERA2 cells are seeded at 1×10⁶ cells per 75 cm2 flask in differentiation medium (growth medium supplemented with 10 μM retinoic acid). The NTERA2 cells commit to neural differentiation within 2-3 days and neurons appear after ˜10 days.

### Example 2

### Human ES Cell Neural Differentiation in Embryoid Bodies (Cell Aggregation)

Logarithmically growing ES cells are resuspended in embryoid body (EB) medium (DMEM knockout, 20% knockout serum replacement, 1% non-essential amino acids, 1 mM β-mercaptoethanol and 1 mM glutamine) and cultured at 37° C. in 5% CO2 in air, in 100 mm Corning Ultra low binding cell culture or bacterial culture dishes to prevent cellular attachment. The medium is replaced every alternate day. After ˜21 days in suspension, differentiated EB's are present. These are plated on a gelatin-coated surface in EB medium at a density of 50 embryoid bodies per 25 cm2. Neural differentiation is visible as outgrowths from the attached embryoid bodies ˜24 hours after re-plating.

### Example 3

### Derivation and Differentiation of Neurospheres from Human ES Cells

Confluent ES cells are resuspended in EB medium and placed in a 25 cm2 cell culture flask at 37° C. in 5% CO₂ in air for 4 days. The medium is changed daily. After ˜4 days the EB's are placed in neurospheres medium consisting of DMEM/F12, N2 supplement, FGF-2, (20 ng/ml), insulin (20 μg/ml) and hepatin sulphate sodium salt (2 μg/ml) and plated into gelatin-coated 25 cm2 flasks. The medium is replaced every alternate day. After ˜10 days neural rosettes are visible. The bacillus-derived neutral metalloprotease Dispase™ (100 μg/ml—Invitrogen) is used to detach disaggregated neural rosettes and these are resuspended in neurospheres medium and dispensed onto 1% agarose in DMEM/F12 coated flasks. The neurospheres are treated with fresh neurospheres medium every 5 days and sub-cultured every 2-3 weeks. For differentiation studies the neurospheres are seeded onto gelatin-coated flasks in neurospheres medium and after several passages the neurospheres-derived cells will be the most dominant cell type in the culture. These cells are normally positive for the early neural markers such as musashi-1 and nestin. The neurospheres can then be efficiently differentiated using method such as those outlined above i.e. Marchal-Vitorion S., et al., 2003, (Cell Neurosci., 24, 198-213) and Zhang, S. C. et al., 2001, (Nat. Biotechnol. 19, 1129-1130).

### Inhibitors of Neural Differentiation

### [1] Adenosine Dialdehyde

S-adenosylmethionine (AdoMet) is a universal methyl donor for a wide range of biological methylation reactions including DNA methylation. Genes can be transcriptionally inactivated by the methylation of CpG loci and this is occasionally associated with cellular differentiation processes for example neural differentiation requires a cascade of genetic programs that control stage-specific gene activities. These activities are controlled not only at the transcriptional level but also by epigenetic modifications including DNA methylation.

Methylation reactions performed by Adomet-dependant methlytransferases leads to the generations of two products, the methylated substrate and the by-product Ado-homocysteine (AdoHcy) which is itself a potent inhibitor of Adomet-dependant methlytransferases. AdoHcy is further broken down to adenosine and homocysteine by the enzyme S-adenosylhomocyteine hydrolase (SAHH).

Inhibition of SAHH therefore results in the accumulation of the methlytransferase inhibitor AdoHcy. Adenosine dialdehyde (AdOx) is a potent inhibitor of SAHH and therefore indirectly of Adomet-dependant methlytransferase and thereby neuronal differentiation

P19 are embryonal carcinoma cells they can be differentiated into neurons by the cell aggregation method in the presence of retinoic acid as described earlier. However, exposure of the cells to AdOx (1 μM) on the first day of the differentiation process decreased i) the number of neurites observed and ii) the expression levels of the neuronal markers β-tubulin, NeuroD1 and mash1. Therefore AdOx via its indirect inhibition of Adomet-dependant methlytransferase interrupts the neuronal differentiation in P19 cells (Hong, S. et al., 2008, Biochem. Biophys. Res. Commum., 377, 935-940).

### [2] D-Theo-1-Phenyl-2-Decanoylamino-3-Morphlino-1-Propanol (D-PDMP)

Gangliosides have been implicated in neural development. Using an in-vitro neuronal differentiation model involving P19 EC cells Liour S. S. & Yu R. K., 2002, (Neurochemical Res. 27, 1507-1512) demonstrated that the ganglioside biosynthetic inhibitor D-theo-1-phenyl-2-decanoylamino-3-morphlino-1-propanol (D-PDMP) inhibited neurite outgrowth, eventually causing the death of P19-derived neurons.

The P19 EC cells (1×10⁶ cells per dish) were cultured in α-MEM (Invitrogen) supplemented with 2.5% foetal calf and 5% calf serum. They were induced to differentiate in the presence of 5 μM retinoic acid in bacterial grade dishes for 4 days, after which they were dispersed in growth medium minus retinoic acid and plated on poly-lysine coated cell culture dishes. The medium was changed every ˜3 days. The ganglioside inhibitor D-PDMP (50 μM) was added 3 days prior to treatment with retinoic acid and maintained throughout the differentiation process. The results indicated that D-PDMP reduced i) the proliferation of the undifferentiated P19 EC cells without any indication of cell death and ii) abolished neurite elongation, note—neurites were present but they failed to develop correctly. By using other ganglioside inhibitors the authors were able to demonstrate that the effect of D-PDMP on neuronal differentiation was not solely related to the inhibition of ganglioside biosynthesis.

### [3] Indocarbazostatins

The Indocarbazostatins A, D, C and D are generated by Streptomyces species and have all been demonstrated to be inhibitors of the NGF-induced neuronal differentiation of rat PC12 cells (Matsuura N et al. 2002, J. Antibiotics 55, 355-362 and Feng, Y. et al., J. Antibiotics 57. 627-633). In brief, PC12 cells were grown in DMEM supplemented with 0.35% glucose, 10% foetal calf serum and 10% horse serum. The PC12 cells were plated into the wells of a 96-well collagen type 1 coated plate. After 12 hours the indocarbazostatins were added and 12 hours later NGF. Neuronal differentiation was monitored by observing the generation of neurite processes.

### [4] Indolocarbazoles

In similar experiments as those described above for the indocarbazostatins-mediated inhibition of NGF-induced neuronal differentiation of rat PC12 cells, the indolocarbazoles K-252a and b have also been demonstrated to be effective inhibitors of neural differentiation (Matsuura N et al. 2002, J. Antibiotics 55, 355-362). These compounds apparent mediate their inhibition of neurite elongation by inhibiting p140 trk tyrosine kinase NGF-receptor.

### [5] 2″-Amino-3″-Methoxyflavone (PD98059)

PD 98059 is a potent, cell-permeable and selective inhibitor of MAPK/ERK kinase 1 (MAP kinase kinase 1 or MEK1). It blocks the activation of MEK1, therefore inhibiting the subsequent phosphorylation and activation of MAP kinase. Pang, L. et al., 1995 (J. Biol. Chem. 270, 13585-13588) demonstrated that PD98059 completely blocks the NGF-induced neurite formation in PC12 cells without affecting cell viability. Indicating that the MAP kinase pathway appears to be critical for NGF-induced neuronal differentiation in PC-12 cells.

### [6][7-(Benzolylamino)-4,9-Dihydro-4-Methyl-9-Oxo-Pyrazolo[5,1-b]Quinazoline-2-Carboxlic Acid] PD90780

The substituted pyrazoloquinazolinone PD90780 interacts with NGF, thereby preventing its binding to the p140 trk tyrosine kinase NGF-receptor and the common neurotrophin receptor p75NTR. Inhibiting the binding of NGF abolishes the NGF-mediated neuronal differentiation of PC12 cells Spiegel K et al. 1995 Biochem. Biophys Res Commun. 217, 488-494.

### [7] AG870

AG-879 is a member of the tyrphostin family of tyrosine kinase inhibitors. It inhibits the p140 trk tyrosine kinase NGF-receptor autophosphorylation selectively with no inhibition of EGF or PDGF receptor phosphorylation (IC50=10 μM). Like the above chemicals AG-879 also inhibits NGF-induced neurite outgrowth in PC12 cells Ohmichi M et al. 1993, Biochemistry 4, 32 4650-4658.

The effect of inhibitors (and chemicals, drugs or cosmetics) such as those described above on neural development can be monitored during progenitor cell differentiation. Progenitor cells are exposed to the inhibitor and the effect on the cellular developmental process will be assessed by monitoring the extent of differentiation. This can be achieved by measuring the expression of cell/tissue specific markers associated with the differentiated cell type by techniques such as quantitative immuno-cytochemistry.

Specific neural cell Markers indicative of neural development include the following, (details of antibodies commercially available from Abcam Inc. are also described).

### Neural Stem Cell Markers—Early Markers

[1] Aggrecan ARGxx (antibody BC-3, ab3773)—Detected in neural precursor cells.
[2] CD133 (antibody 32AT1672, ab5558)—A marker for neural and embryonic stem cells.
[3] Dlx5 (antibody ab54729)—A transcriptional regulator during neural development.
[4] EMX2 (antibody ab11849)—Emx2 is a homeobox protein implicated with Otx1/2 to specify cell destiny in the developing cerebral cortex of the CNS.
[5] Nestin (antibody 10C2, ab22035)—Expressed in early embryonic neuroepithelial stem cells. Nestin is widely used as a marker for stem/progenitor cells, glioma cells.
[6] NeuroD1 antibody (ab60704)—An important differentiation factor in neurogenesis

### Neural Stem Cell Markers—Late Markers

[1] BRN3A (antibody ab30880)—A transcription factor, implicated in the regulation of neuronal genes.
[2] BRN3B (antibody ab32264)—Found in the retina within a subpopulation of ganglion cells where it determines the identity of a subset of visual system neurons.
[3] Musashi 1/Msi1 (antibody ab60600)—Expressed in neural stem cells
[4] NR2E1/Tailless (antibody ab66125)—Expressed in the brain.
[5] Nucleostemin (antibody ab52784)—Found in embryonic and adult CNS stem cells.
[6] Oct6 (antibody ab72681)—A transcription factor involved in early embryogenesis and neurogenesis expressed in embryonic stem cells and in the developing brain.
[7] Pax2 (antibody ab55490)—A transcription factor required during the development of the nervous systems, including the midbrain, hindbrain, spinal cord, eye, ear.
[8] SOX2 (antibody 57CT23.3.4, ab75485)—A transcriptional activator expressed in the developing nervous system.
[9] SOX4 (antibody 154C4a, ab70598)—Transcription factor expressed in the CNS. Expression increases in the developing CNS.
[10] SOX10 (antibody ab27655)—A transcriptional activator, acts as a nucleocytoplasmic shuttle protein, important for neural crest and peripheral nervous system development.
[11] SOX11 (antibody ab50194)—SOX11 is important in the developing nervous system.
[12] SOX22 (antibody 86C2a, ab54371)—A transcriptional activator expressed in foetal brain and kidney and adult heart.
[13] Vimentin (antibody RV202. ab8978)—Neural Stem Cell Marker
[14] CDw338 (antibody BXP-21, ab3380)—Hematopoietic/Neural Stem Cell Marker,

### Neural Crest Cells—Markers

[1] Neurogenin 1 (antibody ab66498)—Transcription factor expressed in a distinct progenitor population. It regulates neuronal development and differentiation.
[2] Neurogenin 2 (antibody ab57560)—Transcription factors that regulates the neocortex development. The transition from cell proliferation to neurogenesis involves Neurogenin 2.
[3] Neurogenin3 (antibody ab54743)—A transcription factors that plays an important role in neurogenesis from migratory neural crest cells.
[4] MASH1 (antibody ab76987)—Expressed in the early development of neural cells. Found in the neuroepithelium of the spinal cord, mid- and ventral-forebrain. Later found the brain.

### Astrocytes—Markers

[1] Astrocyte (antibody 10E4/R5, (ab3268)—Astrocyte Marker
[2] CaMKII (ab63377)—Kinase of the CNS, functions in long term potentiation and neurotransmitter release.
[3] EAAT1 (antibody ab416)—Expressed in the frontal cortex, hippocampus and basal ganglia.
[4] Early CD15 antibody (28, ab20137)—Expressed in astrocytes and certain epithelial cells.
[5] Ganglioside GD3 (antibody MB3.6, ab78361)—All astrocytomas express GD3 antigen.
[6] GFAP (antibody GF5, ab10062) Astrocyte Marker—Expressed in astrocytes. astroglia, satellite cells in peripheral ganglia, Schwann cells and neural stem cells.
[7] S100 (antibody 4C4.9, ab4066) Astrocyte Marker—Localizes in astrocytes, Schwann's cells, ependymomas and astrogliomas.
[8] Survivin (antibody 32.1, ab9178)—Expressed in astrocytes and some neurons.
[9] Other astrocytes markers include
ABCA1 antibody (HJ1, ab66217) & ABCA7 antibody (7A1-144, ab48265)
ALDH1L1 antibody (ab56777)
Thrombospondin antibody (A4.1, ab3131).

### Glial and Microglial Cells—Early Markers

[1] CNTF (antibody 4-68, ab78269)—Expressed in glial cells within the CNS and PNS. CNTF stimulates the differentiation of a variety of neuronal cell types.
[2] Twist (antibody 2C1a, ab50887)—Twist is a transcription factor expressed in gliomas. It may have a role in CNS development and angiogenesis.

### Glial and Microglial Cells—Late Markers

[1] cCD11b (antibody ab8879)—Commonly used as a microglial markers in nervous tissue.
[2] Iba1/AIF1 (antibody ab54749)—A Ca2+ binding peptide expressed by microglial cells.
[3] MRP8 (antibody 2C5/4, ab19860)—Expressed by microglial cells.
[4] Nfasc155 antibody (ab77951)—Nfasc155 in unmyelinated axons in glia
[5] PAX6 (antibody AD2.38, ab78545)—A transcription factor involved in the development of the eye, nose, central nervous system and pancreas.
[6] BLBP (Antibody ab27171)

BLBP can be used as a molecular marker for radial glia (a major neural progenitor cell type and a scaffolding supporting neuronal migration)

### Purkinje Cells—Early Markers

[1] L1 CAM (antibody 2C2, ab24345)—Expressed is in neuroectoderm tissues. Involved in axon growth, neural migration and in mediating neuronal differentiation.

### Purkinje Cells—Late Markers

[1] PTP zeta (antibody ab78019)—Developmentally regulated in the brain and expressed in the CNS, where it is localized in the Purkinje cell layer of the cerebellum, the dentate gyrus, and the subependymal layer of the anterior horn of the lateral ventricle.
[2] NSMase2 (antibody ab68735)—Restricted to neurons, Purkinje cells, pyramidal cells, neurons of the dentate gyrus granular layer, and neurons in the pontine nuclei. Also present in the hypothalamic nuclei, neurons in the piriform cortex, and nuclei of the brainstem.
[3] Aldolase (antibody 1F8, ab67204)—Aldolase C is expressed in the brain and nerves.
[4] Precerebellin (antibody ab36909)—A precursor of the brain-specific cerebellin. The active form is enriched in postsynaptic structures of cerebellar Purkinje cells and in cartwheel neurons of the dorsal cochlear nucleus.
[5] Calbindin (antibody CL-300, ab9481)—A marker for cerebellum Purkinje cells

### Neurons—Early Markers

[1] PROX1 (antibody ab57746)—Plays a fundamental role in early development of the CNS. It regulates gene expression and development of postmitotic undifferentiated young neurons.
[2] CD90 (antibody 1.BB.730, ab62009)—Expressed on neural cells, T cells, early hematopoietic progenitors, fibroblasts, neurons and Kupffer's cells.
[3] UCHL1/3 (antibody ab75275)—A role in the regulation of neuronal development.
[4] PLAGL1 (antibody ab55659)—Expressed in neuron-epithelia during early brain development
[5] HLXB9 (antibody EPR3342, ab79541)—A homeobox gene expressed selectively by motor neurons in the developing vertebrate CNS developmentally regulates neuronal fate.
[6] NeuroD2 antibody (ab66607)—Induces neuronal differentiation and survival.
[7] NEUROD4 antibody (ab67168)—Mediates neuronal differentiation
[8] NEUROD6 antibody (ab77998)—Involved in neuronal differentiation and maturation

### Neurons—Intermediate Markers

[1] NNPTX2 (antibody ab69858)—A neuronal immediate early gene that plays a role in excitatory synaptogenesis.
[2] Neuroglycan C (antibody ab56941)—Involved in neuronal circuit formation in the CNS.
[3] TBR2 (antibody ab58225)—A transcription factor, expressed by intermediate progenitor cells during development. IPCs divide within the ventricular zone (VZ) or subventricular zone (SVZ) and produce a strictly neuronal population

### Neurons—Late Markers

[1] SIRP (antibody OX-41, ab9295)—Expressed by myeloid cells and neurons
[2] Ataxin 7 (antibody ab11434)—located in the cytoplasm and on the nuclear membrane of normal brain neurons.
[3] GIRK2 (antibody ab30738)—Neuronal GIRK2 channels are involved in the regulation of the excitability of neurons and may contribute to the resting potential.
[4] Profilin 2 (antibody ab55611)—Profilin 2 is the neuronal specific.
[5] AP180 (antibody AP180-I, ab11329)—Expression restricted to cells of neuronal origin.
[6] PGP9.5 (antibody ab27053) Neuronal Marker—Expression of PGP9.5 is highly specific to neurons and to cells of the diffuse neuroendocrine system and their tumors.
[7] S or CS1 (antibody ab16641) Neuronal Marker—S or CS1 immunoreactivity is widespread in a population of neurons throughout the brain.
[8] Nova1 (antibody ab77926)—Nova 1 is a neuron-specific RNA-binding protein.
[9] NSE (antibody ab944) Neuronal Marker—Neuron specific enolase is expressed primarily in neurons, in normal and in neoplastic neuroendocrine cells.
[10] HB Hu protein (antibody 16A11, ab14370)—Binds specifically to a conserved peptide epitope present in members of the Hu family of vertebrate neuronal proteins.
[11] ELAVL4 (antibody 16C12, ab14369)—May play a role in neuron-specific RNA processing. It is localised to brain tissue.
[12] SAPAP3 (antibody ab67224)—Located at postsynaptic regions in neuronal cells.
[13] Early Ki67 antibody [PP-67, 526)—Ki67 is routinely used as a neuronal marker.
[14] MAP2 (antibody HM-2, ab11267) Neuronal Marker—MAP2 is the major microtubule associated protein of brain tissue.
[15] Myelin Basic Protein (antibody MBP101, ab62631)—abundant protein components of the myelin membrane. May have a role in early brain development.
[16] Kinesin (antibody ab25715), Kinesin 2 (antibody K2.4, ab24626) and Kinesin 5A (antibody ab5628)—Involved in vesicle transport in neuronal cells. 5A is neuron-specific.
[17] NeuN (antibody A60, ab77315)—Neuron-specific nuclear protein is a marker for neurons. NeuN is found throughout the nervous system, cerebellum, cerebral cortex, hippocampus, thalamus and spinal cord.
[18] Nfasc186 (antibody ab31719)—Expressed in neurons at the Nodes of Ranvier.
[19] Pin1 (antibody ab12107)—Implicated in tau pathologies that underlie Alzheimer's Disease. Pin1 could be pivotal for maintenance of normal neuronal function.
[20] Neuroligin 3 (antibody ab57375)—Neuroligin 3 is a neuronal cell surface protein.
[21] PDGF beta Receptor (antibody Y92, ab32570)—Expressed on neurons.
[22] Cofilin (antibody ab54532)—Cofilin is ubiquitous but expressed especially in neurons.

### Hippochampal Neurons

[1] SynGAP (antibody EPR2883Y, ab77235)—Expressed exclusively at synapses in hippocampal neurons.

### Telencephalic Neurons

[1] Synaptopodin (antibody ab50485)—Essential for the formation of spine apparatuses of telencephalic neurons. Involved in synaptic plasticity.

### Dopaminergic Neurons—Early Markers

[1] PITX3 (antibody ab30734)—The transcription factor regulates the differentiation of dopaminergic neurons
[2] Nurr1 (antibody ab12261)—A transcription factor expressed in the embryonic ventral midbrain. Critical for the development and maintenance of dopamine neurons.
[3] AMSX1 (antibody 4F11, ab73883)—Acts in parallel with the Wnt1 to establish the midbrain dopaminergic progenitor domain, gives rise to a neuronal population.

### Dopaminergic Neurons—Late Markers

[1] Tyrosine Hydroxylase (antibody 185, ab10372) Neuronal Marker—TH has a role in the physiology of adrenergic neurons and is regularly used as a marker for dopaminergic neurons.
[2] Dopamine D2 Receptor (antibody ab30743)—Expressed in the pituitary and brain.
ALDH1A1 (antibody ab23375)—Expressed in the dorsal retina, ventral midbrain (dopaminergic neurons), and hematopoietic stem cells.
[3] DOPA Decarboxylase (antibody ab3905)—An enzyme implicated in the synthesis of the neurotransmitters: dopamine and serotonin

### Cholinergic Neurons

[1] Choline Acetyltransferase (antibody ab54599)—Serves as a specific marker for cholinergic neurons in both peripheral and central nervous systems

### Sensory Neurons

[1] Syntaxin and 2 (antibody 4H256, ab18010 and ab12369 respectively)—Neuronal syntaxins, localized to terminals of sensory neurons and nerve reaching small blood vessels.

### Nociceptive Neurons

[1] Peripherin (antibody 2Q135 ab17999) nociceptive (pain) neuron marker—Found in the neurons of peripheral ganglia and their processes.

### Motor Neurons

[1] Islet 1 (antibody ab20670) Neural Stem Cell Marker—A transcription factor playing a role in neural tube motor neuron differentiation and the embryogenesis of pancreatic islets cells.
[2] Islet 2 (antibody ab26117) Neural Stem Cell Marker—A transcription factor that defines subclasses of motor neurons.

### Pyrimidal Neurons—Early Markers

[1] Emx1 (antibody ab32925)—A homeobox gene specifically expressed pyramidal neurons. Emx1 is a reliable marker of pyramidal neurons and pyramidal cell lineage.
[2] TBR1 (antibody (ab56994)—Expressed in the cerebral cortex. During early embryogenesis it distinguishes the paleocortex, limbic- and neo-cortex domains.

### Pyrimidal Neurons—Late Markers

[1] Hippocalcin (antibody ab24560)—Restricted to the CNS and most abundant in pyramidal cells of the hippocampal CA1 region

### Oligodendrocytes—Early Markers

[1] A2B5 (antibody 2Q162, ab68385)—A cell surface ganglioside epitope expressed in developing oligodendrocyte progenitors and neuroendocrine cells
[2] PDGF alpha Receptor (antibody Y92, ab32570)—Alpha subunit expressed in oligodendrocyte progenitor cells.
[3] Olig1 (antibody ab21943)—Olig1 promotes formation of oligodendrocytes.
[4] Olig2 (antibody ab56643)—Transcription factor required for oligodendrocyte, spinal cord motor neurons, and for the development of somatic motor neurons in the hindbrain.
[5] OSP (antibody ab7474), Oligodendrocyte Marker—Expression is highly regulated during development, it may play a role in the growth and differentiation of oligodendrocytes
[6] Olig3 antibody (ab78006)—Olig3 is transiently expressed in different types of progenitors of embryonic central nervous system.

### Oligodendrocytes—Intermediate Markers

[1] Sortilin (antibody ab16640)—Expressed in brain, spinal cord and muscle. Sortilin acts as a receptor for neurotensin. Sortilin is expressed during embryogenesis.

### Oligodendrocytes—Late Markers

[1] Myelin oligodendrocyte glycoprotein (antibody F3-87-8, ab24022)—MOG is found on the surface of myelinating oligodendrocytes.
[2] CNPase (antibody 11-5B, ab6319) Oligodendrocyte Marker—Expressed by oligodendrocytes and by Schwann cells.
[3] Myelin PLP (antibody plpc 1, ab9311), Oligodendrocyte Marker—The most predominant myelin protein in the CNS. Involved in oligodendrocyte development and axonal survival.
[4] CaMKII (antibody ab63377)—A ubiquitous kinase that is abundant in the brain as a major constituent of the postsynaptic density.

### Neuroendocrine Cells

Chromogranin A (antibody 23A1, ab36997)—Expressed in neuroendocrine cells.

### Axons

[1] Neurofilaments constitute the main structural elements of neuronal axons, sympathetic ganglion cells and dendrites.
200 kD Neurofilament Heavy (antibody ab8135)
160 kD Neurofilament Medium (antibody 3H11, ab7256)
145 kD Neurofilament (antibody 2E30, ab35953)
68 kDa Neurofilament (antibody DA2 ab4572)
[2] 14-3-3 (antibody 2Q248, ab14121)—Localized in neurons, and axonally transported to the nerve terminals.
[3] Fez1 (antibody ab53562)—Involved in axonal outgrowth as a component of the network of molecules that regulate cellular morphology and axon guidance machinery
[4] Dynein heavy chain (antibody 440.4, ab6305) & intermediate chain 1 (antibody 70.1, ab6304) Expressed in microtubules Dynein has been implicated in axonal transport
[5] Gigaxonin (antibody ab27041)—Ubiquitously expressed essential for neuronal function and survival.
[6] Lingo1 (antibody ab23631)—Expressed in mouse and human brains.
[7] MAP1a+MAP1b (antibody HM-1, ab66021) MAP2 (antibody ab32454) MAP1B (antibody 3G5 ab3095), MAP2a+MAP2b antibody (AP20, ab3096)—Microtubules are composed of tubulin and microtubule-associated proteins. MAP1 is neuronal-specific.
[8] Netrin G1 ligand (antibody ab31983)—Expressed highly in thalamic axons in the striatum and the cerebral cortex. NGL1 is implicated in facilitating axonal growth.
[9] Plexin B2 (antibody ab41098)—This receptor has a key role in axon guidance.
[10] Robo2 (antibody ab72972)—A receptor for Slit2 and Slit1, which guide axonal navigation of the neural tube during neuronal development.
[11] Tau (antibody ab8763) Neuronal Marker—Tau is a neuronal microtubule associated protein found predominantly on axons.
[12] Tubulin (antibody YOL1/34, ab6161) Microtubule Marker—The tubulin family are involved in microtubule organization.

### Schwann Cells

[1] NGF Receptor (antibody MLR2, ab61425)—Expressed in Schwann cells and neurons. During development NGFR regulates neuronal growth, migration, differentiation and cell death.
[2] Myelin (antibody pm432B5, ab58513)—Myelin is produced by oligodendrocytes in the CNS and Schwann cells in the peripheral nervous system
[3] Gliomedin (antibody ab24483)—Expressed by myelinating Schwann cells, accumulates at the edges of each myelin segment during development.
[4] Lgi4 (antibody KT18, ab63289)—Expressed in Schwann cells.
[5] Myelin Protein Zero (antibody ab31851)—Expression of MPZ is restricted to Schwann cells. It is the major structural protein of peripheral myelin and nerves.
[6] Lgi4 (antibody KT18. ab63289)—Lgi4 is expressed in Schwann cells, controlling axon segregation and myelin formation

### Dendrites—Early Markers

Arg 3.1 (antibody ab23382)—An immediate early gene, enriched in the brain expression is induced by neuronal activity. Expressed in dendrites in the hippocampus, amygdala, hypothalamus, striatum and cortex

### Dendrites—Late Markers

[1] RRIMS3 (antibody ab50198)—localises to neuronal dendrites and the postsynaptic densities.
[2] Drebrin (antibody M2F6, ab12350)—Drebrin is a major neuronal F-actin binding protein involved in the control of actin dynamics and neuronal morphogenesis.
[3] Neuron specific beta III Tubulin (antibody ab18207)—Abundant in the CNS and PNS where it is expressed during fetal and postnatal development.
[4] SAP102 (antibody 7D3 mAb 119, ab69738)—Synapse-associated protein 102 is detected in dendritic shafts and spines of asymmetric type 1 synapses.
[5] Others—Follicular dendritic cell marker (antibody ab8138)
Dendritic cell antibody (antibody ab8171)

### Growth Cone—Early Markers

[1] CRMP1 (antibody ab76995)—CRMP2 (antibody ab54546) CRMP5 (antibody ab77158)
Collapsin response mediator proteins are involved in neuronal differentiation, axonal and growth cone guidance during neural development.
[2] NRP2 (antibody 96009, ab50205)—NRP2 is a member of the neuropilin family of receptor proteins, may play a role in cardiovascular development and axon guidance.

### Growth Cone—Late Markers

[1] Agrin (antibody AGR 131, ab12362)—Promotes clustering of nicotinic acetylcholine receptors (and others) during development at the neuromuscular junction
[2] BAI1 associated protein 2 isoform 3 (antibody ab791)—Brain-specific angiogenesis inhibitor
[3] BAIAP2 (antibody ab56588)—A brain-specific angiogenesis inhibitor, involved in neuronal growth-cone guidance.
[4] BASP1 (antibody ab79349)—A protein abundantly expressed in the brain.
[5] Doublecortin (antibody ab28941)—A microtubule binding protein found in cell bodies and leading processes of migrating neurons and axons of differentiating neurons.
[6] Eph receptor A1 protein (antibody ab55900), A2 (antibody RM-0051-8F21 ab73254), A3 (antibody 6C1B6, ab76361), A4 (antibody 7D3D4 ab70403), A5 (antibody ab54633), A6 (antibody ab58022), A7 (antibody ab54640), A8 (antibody ab10615), B1 (antibody 5F10A4, ab66326), B2 (antibody ab54650), B3 (antibody ab54717), B4 (antibody 4A12G8, 5G2F8, ab66336) and B6 (antibody 2A6B9, ab66325)—EPH-related receptors have been implicated in mediating neural tissue developmental events. Developing and adult neural tissue express all of the Eph receptors and ephrin ligands. Role of the EPH-receptors is in mediating axonal guidance and neural crest cell migration.
[7] Ephrin A1 (antibody ab7040), A2 (antibody ab65041), A3 (antibody ab66150), A4 (antibody ab53062), B2 (antibody ab75868) and B3 (antibody ab53063)—Ephrins are the ligands of the Eph receptors implicated in mediating developmental events in the nervous system.
[8] GAP43 (antibody GAP-7B10, ab50608)—A neuronal growth cone protein.
[9] GPRIN1 (antibody ab74577)—GPRIN1 is involved in neurite outgrowth
[10] LIM kinase 1 (antibody ab51200)—Active in the brain and spinal chord, where it is believed to be involved in the development of nerve cells.
[11] NCAM (antibody 123C3, ab28377)—Expressed on most neuroectodermal derived cells.
[12] Neuroserpin (antibody ab55587)—Predominantly expressed by neurons of the developing and adult brain. This serpin is secreted from axonal growth cones of the CNS and PNS.

### Soma

[1] ALK (antibody ALKc ab650)—ALK is found in the nervous system expressed in forebrain neurons.
[2] Membralin (antibody ab21818)—Expressed in the central nervous system.
[3] Necdin (antibody ab55501)—Brain specific growth suppressor
[4] STEP (antibody 23E5, ab16967)—Is a neural-specific protein-tyrosine phosphatase.

### Synapse—Early Markers

Syntenin (antibody ab19903)—Regulated developmental profile in neurons and is most abundant in the period of intense growth and synapse formation.

### Synapse—Late Markers

[1] EAAT2 (antibody ab77039)—Essential for terminating the postsynaptic action of glutamate.
[2] Neurexin II alpha (antibody ab34245), Neurexin 1 beta (antibody ab77596) and NRXN3 (antibody ab18523)—Neuronal proteins that function as cell adhesion molecules during synaptogenesis
[3] Amphiphysin (antibody C14-23, ab16770)—Associated with the cytoplasmic surface of synaptic vesicles.
[4] Bassoon (antibody SAP7F407, ab13249)—Localized to the presynaptic nerve terminals.
[5] SAP102 (antibody ab12086)—A synapse protein, detected in dendritic shafts and spines of asymmetric type 1 synapses.
[6] CASK (antibody ab11343)—Localized to neuronal synapses.
[7] CPLX1 (antibody ab15855) and CPLX2 (antibody ab77978)—Cytosolic proteins that function in synaptic vesicle exocytosis.
[8] CRIPT (antibody ab16422)—Colocalises with PSD95 in the postsynaptic density of excitatory synapses throughout the brain, but is not detected in inhibitory synapses.
[9] CSP (antibody ab79346)—Localized to the cytoplasmic surface of the synaptic vesicle.
[10] CTBP2 (antibody ab67161)—Acts as a scaffold for specialized synapses
[11] Dystrobrevin alpha (antibody ab72793)—Localizes to the sarcolemma may be involved in the formation and stability of synapses.
[12] HOMER2 (antibody ab75037)—Plays an important role in maintaining the plasticity at glutamatergic synapses.
[13] HOMER3 (antibody ab75038)—A post-synaptic density scaffolding protein.
[14] ICA1 (antibody ab55253)—May play a role in neurotransmitter secretion, expressed abundantly in pancreas, heart and brain
[15] Munc 13 (antibody ab27077)—Involved in priming synaptic vesicles.
[16] Munc18 (antibody ab3451)—Regulates synaptic vesicle docking and fusion. It is essential for neurotransmission and binds syntaxin.
[17] Neuroglycan C (antibody ab31946)—Expressed in the central nervous system.
[18] Neuroligin 1 (antibody ab56882), Neuroligin 2 (antibody ab36602)—Neuroligins are synaptic cell-adhesion molecules.
[19] PSD93 (antibody ab12097) Synaptic Marker—Clusters the N-methyl-D-aspartate receptor (NMDAR) at synapses.
[20] PSD95 (antibody 6G6-1C9, ab2723)—Located at postsynaptic sites to form a scaffold for the clustering of receptors, ion channels, and associated signalling proteins
[21] Piccolo (antibody ab20664) Synaptic Marker—A presynaptic cytomatrix protein concentrated at the presynaptic side of synaptic junctions.
[22] RIC8 (antibody ab24383)—Positively regulate synaptic transmission.
[23] SAP97 (antibody RPI 197.4, ab69737)—Membrane associated synapse protein facilitates ion channel clustering at synaptic terminal.
[24] SAPAP3 (antibody ab67224)—Localized at postsynaptic density (PSD) in neuronal cells
[25] SNAP23 (antibody ab57961)—Synaptosomal Associated Proteins play a key role in the process of membrane fusion in intracellular vesicle trafficking.
[26] SNAP25 (antibody ab66066)—A pre-synaptic nerve terminal protein that plays an essential role in synaptic vesicle fusion and exocytosis.
[27] SNAP29 (antibody ab56566)—Involved in membrane trafficking steps, binds to syntaxins.
[28] SNAPIN (antibody ab37496)—A component of the SNARE complex required for synaptic vesicle docking and fusion.
[29] SV2A (antibody 15E11, ab49572), SV2B (antibody ab68025) and SV2C (antibody ab33892)—Integral membrane glycoproteins present in all synaptic vesicles.
[30] SYNPR (antibody ab75053)—A component of the synaptic vesicle membrane and thought to play an important role in synaptic vesicle trafficking.
[31] Synapsin I, II and III (antibody ab57468), (antibody EPR3277, ab76494) and antibody ab68849) respectively. Synapsins are neuronal phosphoproteins which associate with the cytoplasmic surface of synaptic vesicles.
[32] Synaptobrevin (antibody 4E240, ab18013)—Involved in the docking and/or fusion of synaptic vesicles with the presynaptic membrane.
[33] Synaptojanin (antibody ab19904) Synaptic Marker—Expressed in the nervous system.
[34] Synaptophysin (antibody 4E206, ab18008)—Present in the membrane of neuronal presynaptic vesicles in brain, spinal cord, retina, vesicles of adrenal medulla, neuromuscular junctions.
[35] Synaptotagmin (antibody ASV30, ab13259)—Integral membrane protein of synaptic vesicles.
[36] Utrophin (antibody DRP3/20C5, ab49174—Located at the neuromuscular synapse and myotendinous junctions, participates in post-synaptic membrane maintenance and receptor clustering.
[37] VAMP2 (antibody clone 3E5, ab53407)—A small integral membrane protein found specifically in synaptic vesicles in neurons.
[38] Synaptotagmin XII (antibody ab76261)—Involved in the regulation of transmitter release in the nervous system, serves as a Ca(2+) sensors in vesicular trafficking and exocytosis.

### Other Neural Markers

[1] MEF2A (antibody ab55547)—Abundant in granule neurons of the cerebellar cortex throughout synaptogenesis. Also has key roles in cardiac and skeletal muscle development.
[2] MEF2B (antibody ab55565)—Regulates the expression of muscle related genes during development. They are also involved in differentiation of certain neurogenic cells.
[3] MEF2C (antibody ab43796)—Controls cardiac morphogenesis and myogenesis. It may also be involved in neurogenesis and in the development of cortical architecture.
[4] MEF2D (antibody ab43797)—Involved in the differentiation of myogenic and also some neurogenic cells.

### Adipocyte Differentiation

Protocols to achieve i) differentiation, ii) inhibitor details and iii) adipocyte markers

The differentiation of progenitor cells into adipocytes is possible utilizing well characterized and published protocols such as that described by Dani C et al., (1997) J. Cell Sci 110, 1279-1285 Using this and related protocols it is possible to generate adipocytes from pluri- and multi-potent progenitor stems cells.

Embryonic stem cells are pluripotent cells and can be maintained in an undifferentiated state in the presence of leukaemia inhibitory factor (LIF). Removing LIF and adding the appropriate differentiation agent results in the commitment of ES cells into a variety of cell types including adipocytes, cardiac cells, skeletal muscle cells and neurons. Adipocytes arise from mesodermal stem cells, a common precursor for myocytes, chondrocytes and osteocytes. Once committed to the adipocytes lineage, pre-adipocytes mature into adipocytes during the later stages of the differentiation process.

Therefore two distinct phases exist in adipogenesis i) between days 2-5 after embryoid body formation, this stage requires retinoic acid and is the committal stage for ES cell derived adipogenesis and ii) corresponding to the terminal differentiation stage. This latter stage requires the adipogenic factor PPARγ. Pre-adipocyte cell lines such as 3T3L1 and 3T3F442A have been used to study the mechanisms associated with the later stages of adipogenesis, resulting in the identification and isolation of several adipocyte-specific genes.

Therefore the early stages of adipogenesis are retinoic acid-dependent and the later stages PPARγ-dependent.

Adipocytes derived from ES cells can be generated after early exposure to retinoic acid followed by the application of classical adipogenic inducers. Under these conditions, large clusters of mature adipocytes are present in 70-80% of the embryoid bodies. Retinoic acid influences the pattern of ES cell differentiation in a time and concentration-dependent manner. During adipocytes differentiation, retinoic acid treatment stimulates the early commitment of ES cells to adipocytes but acts as an inhibitor in the later stages of pre-adipocyte maturation. This later inhibitory effect is due to the repressor action of retinoic acid on the expression of the important adipocyte transcriptional regulator genes PPARδ and C/EBP (Shao, D. and Lazar, M. A., 1997, J. Biol. Chem., 272, 21473-21478).

ERK signalling appears important for adipogenesis as co-treatment of ES cells with retinoic acid and PD98059 9 (a specific inhibitor of the ERK pathway) prevents adipocyte formation. Application of PD98059 has no effect on the differentiation of ES cells into neuron or cardiomyocytes (Bost F., et al., 2002 Biochem J., 361, 621-627).

Differentiation of Embryonic Stem Cells into Adipocytes In Vitro

The mouse embryonic stem cell lines ZIN40, E14TG2a and CGR8 were used by Dani C et al., (1997) J. Cell Sci 110, 1279-1285 to generate adipocytes. Briefly, this process involved culturing the undifferentiated cell lines in feeder-free conditions on gelatin-coated plates in cultivation medium (MEM/BHK21 medium containing 0.25% sodium bicarbonate, ×1 MEM essential amino acids, 2 mM glutamine, 1 mM pyruvate, 100 μM mercaptoethanol and 10% v/v foetal calf serum). Leukemia-inhibitory factor (100 units/ml) was added to inhibit differentiation. In order to differentiate the ES cells into adipocytes, the cells were cultivated as aggregates in embryoid bodies. Each hanging drop contained 1,000 cells in 20 μl cultivation medium. These were maintained for 2 days on the lids of bacteriological plates filled with PBS. The embryoid bodies formed were re-suspended in cultivation medium supplemented with retinoic acid (0.1% v/v) and maintained on bacteriological plate lids. The embryoid bodies were maintained for several days and then allowed to settle onto gelatin-coated plates resuspended in differentiation medium which consisted of cultivation medium supplemented with 85 nM insulin, 2 nM tri-iodothyronine and 10% foetal calf serum.

After 10-15 days cell clusters appear that are filled with lipid droplets. These can be stained using oil red O (a specific stain for triglycerides). The results indicated that 60% of the embryoid bodies formed adipocyte positive colonies as determined by the expression of the adipogenic markers adipsin and PPARγ.

A modification of the Dani, C., et al., (1997), J. Cell Sci., 110, 1279-1285 method was utilised by Rosen, E. D., et al., (1999), Molecular Cell. 4, 611-617 during the differentiation of ES cells (2 day old) derived from control and PPARγ deficient mice. The actual modified protocol briefly, involved the generation of embryoid bodies from embryonic stem cells cultivated in medium containing retinoic acid. The embryoid bodies were then transferred to gelatin-coated six well plates and exposed to 5 μg/ml insulin. On day 17 the embryoid bodies were exposed to dexamethasone (400 ng/ml) and the PDE inhibitor methylisobutylxanthine (500 nM) for 2 days. After which the embryoid bodies were re-suspended in insulin-containing cultivation medium for a further 10 days. This protocol resulted in 70-90% of the cells in the embryoid bodies expressing an adipocyte phenotype as determined by Oil Red O straining of neutral lipids. The wildtype ES cells expressed the early markers adipsin and PPARγ 4 days after the initiation of the differentiation process.

A comprehensive guide to the differentiation of mouse embryonic stem cell and of human adult stem cell into adipocytes is described in Wdziekonski B, Villageois P, and Dani (2007) Curr. Protoc. Cell Biol. Chapter 23: Unit 23.4. This includes protocols required to differentiate mouse, human multi-potent adipose-derived and human mesenchymal stem cells.

### Inhibitors of Adipogenesis

### [1] Inhibition of ERK Signalling

The extracellular-signal regulated kinases (ERKs) are involved in signalling cascades that regulate a number of cellular functions such as cell proliferation and differentiation. Erk-mediated phosphorylation of PPARγ apparently inhibits adipogenesis. PD98059 is a specific inhibitor of MEK1 (the enzyme responsible for ERK activation). The co-treatment of differentiating ES cells with retinoic acid and PD68059 prevented both adipocyte formation and the expression of adipogenic markers in ES cells (Bost F., et al., 2002, Biochem J., 361, 621-627).

### [2] HIV Protease Inhibitors

Therapies employing HIV protease inhibitors are associated with changes in fat metabolism. Lenhard, J. M., et al., (2000), Antiviral Res., 47, 121-129 studied the affect of these inhibitors on adipocyte differentiation using C3H10T1/2 mesenchymal stem cells. In these cells adipogenesis was induced by adding 200 nM insulin and 1 μM of the PPARγ and RXR agonist BRL49653 and LGD1069 respectively.

Under these condition the HIV protease inhibitors nelfinavir, saquinavir and ritonavir reduced the adipocyte differentiation of the mesenchymal stem cells as determined by a reduction in lipogenesis, oil red O-staining and the expression of adipose cell markers AP2 and LPL.

Vernochet, C., et al., (2003), AIDS, 17, 2177-2180 extended this study and assesses the effect of a similar range of HIV protease inhibitors on the adipocyte differentiation of four mouse pre-adipocyte cell lines (3T3-F442A, 3T3-L1, Ob1771 and embryonic stem cells). The method of differentiation was similar to that described by Dani, C., et al., (1997), J. Cell Sci., 110, 1279-1285.

The protease inhibitors nelfinavir, lopinavir inhibited adipocyte differentiation in all cells tested whilst indinavir, saquinavir and ritonvir inhibited the differentiation of only 3T3-L1 and 3T3-F442A cells. The authors concluded that HIV protease inhibitors inhibit adipocyte differentiation depending on the cell model system used.

### [3] Glycogen Synthase Kinase 3 Inhibitors

The signalling events involved in the commitment of stem cells to the adipogenic pathway has not yet been fully characterised. Recently, Mointeiro, M. C., et al., (2009), Stem Cells Dev., 18, 457-463 have shown using mouse embryonic stem cells and the early treatment of retinoic acid that the activation of the retinoic acid receptor is both sufficient and necessary for the commitment of ES cells to adipocytes differentiation. The authors also demonstrated that the retinoic acid receptor beta-induced adipogenesis in ES cells could be abolished by GSK3 inhibitors.

The effect of inhibitors (and chemicals, drugs or cosmetics) such as the HIV protease inhibitors described above etc., on adipocyte development can be monitored during progenitor stem cell differentiation. Progenitor cells are exposed to the inhibitor and the effect on the cellular developmental process will be assessed by monitoring the extent of differentiation. This can be achieved by measuring the expression of cell/tissue specific markers associated with the differentiated cell type by techniques such as quantitative immuno-cytochemistry.

Markers indicative of adipocyte development include the following, (the source of antibodies commercially available from Abcam Inc. are also described).

### Adipocytes—Early Markers

C20orf3 (antibody ab69162)—Involved in adipocyte differentiation
FNDC3B (antibody ab69854)—A positive regulator of adipogenesis
Adiponectin (antibody 19F1, ab22554)—Adipose cells produce and secrete Adiponectin during adipocyte differentiation.
NOC3L (antibody ab74151)—Functions as an adipocyte differentiation accelerating factor.
AE binding protein 1 (antibody ab54820)—Adipocyte enhancer binding protein 1, a transcriptional repressor which binds to the adipocyte enhancer 1 regulatory sequence.
PPAR alpha (antibody ab8934), gamma (antibody ab12409) and delta (antibody ab23673)—All are believed to be involved in adipocyte differentiation.
CEBP Alpha (antibody EP708Y, ab40761) and Beta (antibody A16ab18336-Important adipocyte transcriptional regulator genes.
Adipsin/Factor D (antibody ab8841)—adipocyte specific

### Adipocytes—Late Markers

Leptin (antibody ab3583)—Adipose cells produce and secrete Leptin
Lipoprotein lipase (antibody LPL.A4, ab21356)—Produced and secreted by adipose cells
AEBP2 (antibody 2012C4a, ab74517)—AE (adipocyte enhancer) binding protein 2.
FABP4 (antibody ab37458)—The major fatty acid binding protein found in adipocytes.
FABP5 (antibody ab37267)—The fatty acid binding proteins FABP-4 FABP5 are closely related and expressed in adipocytes.
PDE3B (antibody ab42091)—Expressed in adipocyte tissue.
KIAA1881 (antibody ab78602)—Involved in triacylglycerol packaging into adipocytes.
Resistin (antibody ab3423)—Adipocyte Secreted Factor, is a cytokine that is specifically secreted by adipocytes.
Perilipin A (antibody ab61682)—Found exclusively at the surface of lipid droplets in adipocytes and steroidogenic cells.
Perilipin B (antibody ab3527)—Expression is limited to adipocytes and steroidogenic cells.
Glucose Transporter GLUT4 (antibody ab654)—The stimulation of glucose uptake by insulin in adipose tissue requires translocation of the GLUT4 intracellular sites to the cell surface.
TUG (antibody 4A11A6G11, ab32007)—A putative tether modulating GLUT4 distribution.
Glycerol 3 Phosphate Dehydrogenase antibody (ab34492) Expressed by terminally differentiate adipocytes

### Detection and Quantification

### Cell Imaging

Using a cell imager (for example the IN Cell Analyzer, GE Healthcare) in multiplexing mode, different fluors (e.g cyanine dyes, GE Healthcare) tethered to different specific antibodies can be detected. These fluors are used to detect and measure several target molecules as ES undergo differentiation along a particular path. Thus the instrument can be used to measure, using quantitative immunocytochemistry, the effect of a toxic chemical on up to three different markers from the same sample, thus enabling a toxicity profile of the chemical to be constructed. Furthermore, the method described in this document has an additional feature of discriminating between early and late embryonic stem cell selective biomarkers using techniques well known to the skilled person (e.g. quantitative immunocytochemical, reporter gene assays or RT-PCT and microarray analysis).

As the progenitor cells undergo differentiation these reporters can simultaneously detect and measure several cell/tissue specific molecules present not only in the differentiated cell type but in the original progenitor cell. In addition the ability to generate quantitative data (whether derived from immunocytochemistry or gene reporter assays etc) may allow the determination of sub-lethal dosage levels. This may be of significant value for the drug development or cosmetic industries.

Whilst the present invention has been described in accordance with various aspects and preferred embodiments, it is to be understood that the scope of the invention is not considered to be limited solely thereto and that it is the Applicant's intention that all variants and equivalents thereof also fall within the scope of the appended claims.

1. A method of predicting the toxicity of a chemical on a developmental pathway in a sample comprising the steps of:
(i) treating a control population of undifferentiated stem cells in a sample with an agent to produce a first control population of differentiated cells in a first developmental pathway;
(ii) measuring the levels of at least two biomarkers expressed in said control population of undifferentiated stems cells and/or said first control population of differentiated cells to determine control levels of expression, wherein at least one of said biomarkers is expressed at an early stage of a developmental pathway and/or differentiation and at least one of the biomarkers is expressed at a late stage of a developmental pathway and/or differentiation;
(iii) exposing a test population of undifferentiated stems cells in said sample to a chemical either before or after treating with said agent to produce a first test population of differentiated cells in a first developmental pathway;
(iv) measuring the levels of said at least two biomarkers in said test population of undifferentiated stem cells and/or said first test population of differentiated cells to determine test levels of expression; and
(v) comparing said control levels of expression with said test levels of expression, wherein a difference in the levels of expression following exposure to said chemical is indicative of the toxicity of the chemical on said developmental pathway.

2. The method of claim 1, wherein step (i) comprises the step of
treating a population of undifferentiated stem cells with an agent to produce an n^{th} population of differentiated cells in an n^{th} developmental pathway; and
repeating steps (ii) to (v) to determine a difference in the control levels of expression with the test levels of expression in said n^{th} population, wherein a difference in the levels of expression following exposure to the chemical is indicative of the toxicity of the chemical on said n^{th} developmental pathway.

3. The method of claim 2, wherein said first and said n^{th} developmental pathway are networked developmental pathways.

4. The method of claim 1, wherein step (i) comprises the step of
treating a population of undifferentiated stem cells with an agent to produce a plurality of populations of differentiated cells in a plurality of developmental pathways; and
repeating steps (ii) to (v) to determine a difference in the control levels of expression with the test levels of expression in said plurality of populations, wherein a difference in the levels of expression following exposure to the chemical is indicative of the toxicity of the chemical on said plurality of developmental pathways.

5. The method of claim 4, wherein said plurality of developmental pathways are networked developmental pathways.

6. The method of claim 1, wherein said stem cell is a pluripotent stem cell.

7. The method of claim 6, wherein said pluripotent stem cell is an embryonic stem cell.

8. The method of claim 7, wherein the pluripotent stem cell is an induced pluripotent stem cell.

9. The method of claim 7, wherein pluripotent stem cell is a primordial germ cell.

10. The method of claim 1, wherein said stem cell is an adult stem cell.

11. The method of claim 1, wherein the stem cell is a human stem cell.

12. The method of claim 1, wherein the undifferentiated stem cells comprise different reporter genes operably linked to at least two or more biomarkers, the levels of the two or more biomarkers being quantified by measurement of the different gene products.

13. The method of claim 12, wherein said reporter genes are selected from the group consisting of nitro-reductase, β-galactosidase, β-lactamase, luciferase and fluorescent protein reporter genes.

14. The method of claim 1, wherein the levels of the two or more biomarkers are quantified by a method selected from the group consisting of quantitative RT-PCR, quantitative immunocytochemistry, surface plasmon resonance and microarray analysis.

15. The method of claim 1, additionally comprising determining cell proliferation after steps (iii).

16. The method of claim 1, wherein the method is a multiplex method.

17. A method of predicting changes in a cellular biomap or developmental pathway during human foetal development using the method of claim 1.

18. A kit for carrying out the method of claim 1, comprising means for quantifying the at least two biomarkers and instructions for conducting said method.

19. The kit of claim 18, wherein said means are selected from the group consisting of an antibody, an enzyme substrate and an oligonucleotide primer.
